(19) United States
(12) Patent Application Publication
(10) Pub. No.: US 2026/0278330 A1
(43) Pub. Date: Sep. 17, 2026

(54) DEEP HARMONIC FINESSE: SIGNAL SEPARATION IN WEARABLE SYSTEMS WITH LIMITED DATA

(71) Applicant: THE REGENTS OF THE UNIVERSITY OF CALIFORNIA, Oakland, CA (US)

(72) Inventors: Soheil Ghiasi, Davis, CA (US); Mahya Saffarpour, Karaj (IR)

(73) Assignee: THE REGENTS OF THE UNIVERSITY OF CALIFORNIA, Oakland, CA (US)

(21) Appl. No.: 19/675,157

(22) Filed: May 12, 2026

Related U.S. Application Data
(63) Continuation of application No. PCT/US2025/014661, filed on Feb. 5, 2025.
(60) Provisional application No. 63/550,021, filed on Feb. 5, 2024.

(51) Int. Cl. G06N 3/04; G06F 17/14

(52) U.S. Cl. CPC G06N 3/04; G06F 17/14

(57) ABSTRACT

A method for separation of non-stationary quasi-periodic signals when limited data is available is described. The method utilizes prior knowledge of time-frequency patterns in the signals to mask and in-paint spectrograms. In one implementation this is achieved through an application-inspired deep harmonic neural network coupled with an integrated pattern alignment component. The network's structure embeds the implicit harmonic priors within the time-frequency domain, while the pattern-alignment method transforms the sensed signal, ensuring a strong alignment with the network.

### CROSS-REFERENCE TO RELATED APPLICATIONS

This application claims priority to, and is a 35 U.S.C. § 111(a) continuation of, PCT international application number PCT/US2025/014661 filed on Feb. 5, 2025, incorporated herein by reference in its entirety, which claims priority to, and the benefit of, U.S. provisional patent application Ser. No. 63/559,021 filed on Feb. 5, 2024, incorporated herein by reference in its entirety. Priority is claimed to each of the foregoing applications.

The above-referenced PCT international application was published as PCT International Publication No. WO 2025/171056 A1 on Aug. 14, 2025, which publication is incorporated herein by reference in its entirety.

### STATEMENT REGARDING FEDERALLY SPONSORED RESEARCH OR DEVELOPMENT

This invention was made with Government support under Grant No. 1838939, awarded by the National Science Foundation. The Government has certain rights in the invention.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to methods of signal separation, and more particularly to signal separation in systems providing limited data.

### 2. Background Discussion

Single-detector signal separation methods can be broadly classified into three categories: (1) analytical component decomposition methods with or without consideration of the periodic behavior, (2) deep learning methods that require ample datasets, and (3) deep prior learning methods.

Analytical methods include: both Empirical Mode Decomposition (EMD) and Variational Mode Decomposition (VMD) decompose a time-domain signal into Intrinsic Mode Functions (IMFs), representing different sources. EMD considers the signal's local extremas for extracting IMFs while VMD defines a variational problem assuming each source to have a compact frequency representation around a central frequency which tends to provide improved frequency separation than EMD. Non-negative Matrix Factorization (NMF) operates in both time and frequency domains. In time-frequency, it breaks down the 2-D matrix into two product matrices, which can be interpreted as distinct sources.

Presented for use in sound signal separation in the time-frequency domain, REpeating Pattern Extraction Technique (REPET) operates under the assumption that a sound signal can be represented as sum of a repeating background with a varying foreground. An extension of REPET, REPET-Extended, can adapt to changes in the repeating structure over time, providing better separation quality when the repeating parts of the signal are non-stationary.

Deep learning methods: Due to availability of large datasets in audio applications, a vast body of research has studied deep learning audio source separation methods, which are not applicable when only limited training data is available.

Deep Priors: The structure of a generative convolutional deep neural network, f_{θ}, can be used as a prior for image denoising, in-painting and super-resolution tasks using only one sample in the dataset. The network receives a random vector, z, as input, and is subsequently trained to generate an output that is ‘similar’ to a single, given noisy, low-quality or masked image, x₀. More formally, the training minimizes the cost function min_{θ}E(f_{θ}(z);x₀), where E is a task specific term.

However, all these mechanisms have significant shortcomings when used for collected data, such as in wearable systems, in which there is a limited amount of data available to perform separation of non-stationary quasi-periodic signals.

Accordingly, a need exists for a new mechanism for performing signal separation in systems having small data sets. The present disclosure fulfills those needs and provides additional benefits over existing systems.

### BRIEF SUMMARY

Wearable systems hold significant potential for improving wellness and quality of life for users. In some wearable systems, the collected sensor data is impacted by multiple quasi-periodic non-stationary artifacts that may be considerably stronger than the signal of interest. Examples arise in tissue sensing applications, such as tissue oximetry or blood glucose monitoring, in which the sensor output signal is influenced by a parameter of interest as well as other quasi-periodic physiological phenomena, such as heart rate, respiration and Mayer waves. As obtaining large amount of high-quality data is expensive, and often impractical, successful development of such applications relies on isolation of the target signal from the sensed signal using limited available data.

This disclosure describes a method, referred to herein as Deep Harmonic Finesse (DHF), for separation of non-stationary quasi-periodic signals when limited data is available. Although the disclosure is applicable to a range of applications requiring signal separation, it will be noted that the problem often arises in wearable systems in which a combination of quasi-periodic physiological phenomena give rise to the sensed signal, and excessive data collection is prohibitive. The approach of the present disclosure utilizes prior knowledge of time-frequency patterns in the signals to mask and to in-paint spectrograms. In one embodiment, this is achieved through an application-inspired deep harmonic neural network coupled with an integrated pattern alignment component. The network's structure embeds the implicit harmonic priors within the time-frequency domain, while the pattern-alignment method transforms the sensed signal, ensuring a strong alignment with the network.

The effectiveness of the approach is demonstrated in the context of non-invasive fetal monitoring using both synthesized and in-vivo data. When applied to the synthesized data, the present disclosure exhibits significant improvements in signal-to-distortion ratio (26% on average) and mean-squared-error (80% on average), compared to the best competing method. When applied to in-vivo data captured in pregnant animal studies, the present disclosure improves the correlation error between estimated fetal blood oxygen saturation and the ground truth by 80.5% when compared to the state of the art.

The disclosure also describes extending the DHF method and numerous results, and introduces a Harmonic Priors Ensemble (HarPE) approach. HarPE is a method of integrating a deep ensemble learning framework with a harmonic-aware deep prior neural model to separate the signals. HarPE iteratively masks interference in time-frequency space, and judiciously generates values for the masked regions through which, the target signal of interest can be restored with minimal distortion. The innovative strategy of HarPE includes gradual error correction, balanced optimization of harmonic pixels, and consistent enforcement of bio-pulsation patterns, through which the method exhibits state of the art performance in both estimated signal accuracy and learning stability.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE DRAWINGS

The technology described herein will be more fully understood by reference to the following drawings which are for illustrative purposes only:

FIG. 1A and FIG. 1B is a block diagram of signal separation in the Deep Harmonic Finesse (DHF) procedure, according to at least one embodiment of the present disclosure.

FIG. 2 are diagrams of dilated harmonic convolution and the structure of SpAc LU-Net, utilized, according to at least one embodiment of the present disclosure.

FIG. 3A and FIG. 3B are rendered images depicting in-painting performance comparisons of different convolutional kernels with the same network structure, according to at least one embodiment of the present disclosure.

FIG. 4 are contrast enhanced images of time-frequency spectrogram of the synthesized mixed signals dataset generated for signal separation, according to at least one embodiment of the present disclosure.

FIG. 5A and FIG. 5B are graphs of an SDR comparison and a synthesized signal separation example by DHF, both shown according to at least one embodiment of the present disclosure.

FIG. 6A through FIG. 6C are diagrams of TFO data acquisition and comparisons of fetal signal separation and SpO₂ estimation using DHF according to the present disclosure as compared with a state of the art system.

FIG. 7 are contrast enhanced images of in-vivo DHF fetal signal separation results according to at least one embodiment of the present disclosure.

FIG. 8 is a mixed signal spectrogram from TFO in-vivo sheep study, obtained according to at least one embodiment of the present disclosure.

FIG. 9A and FIG. 9B is a block diagram overview of a signal separation and DHF procedure, according to at least one embodiment of the present disclosure.

FIG. 10A and FIG. 10B depict a pattern alignment procedure in time domain with respect to a specific target signal, according to at least one embodiment of the present disclosure.

FIG. 11 is a block diagram of dilated harmonic convolution and the structure of SpAc LU-Net, according to at least one embodiment of the present disclosure.

FIG. 12 is a block diagram of phase interpolation of the STFT data, according to at least one embodiment of the present disclosure.

FIG. 13A through FIG. 13E are contrast enhanced images of cyclic phase interpolation method applied on pattern aligned time-frequency domain, according to at least one embodiment of the present disclosure.

FIG. 14A and FIG. 14B are plots of SDR comparison versus best previous method and showing Synthesized signal separation example by DHF, according to at least one embodiment of the present disclosure.

FIG. 15A and FIG. 15B are graphs of DHF performance, according to at least one embodiment of the present disclosure.

FIG. 16A and FIG. 16B are contrast enhanced images and graphs of DHF performance improvement, according to at least one embodiment of the present disclosure.

FIG. 17A through FIG. 17D are contrast enhanced images of DHF fetal signal separation on sheep data 2.

FIG. 18A and FIG. 18B are diagrams of Transabdominal Fetal Oximetry (TFO) and an example spectrogram of the acquired PPG, Heart Pulsation (HP), and Respiratory Pulsation (RP) signals, according to at least one embodiment of the present disclosure.

FIG. 19 is a block diagram showing an overview of the a HarPE method according to at least one embodiment of the present disclosure.

FIG. 20A through FIG. 20D is a block diagram of application-inspired deep prior processing elements of pattern alignment, background reduction, and dilated harmonic convolution, according to at least one embodiment of the present disclosure.

FIG. 21 is a block diagram of weak deep learner ‘r’ and final separated unwarped signal generation by summing all boosting weak learners' output, according to at least one embodiment of the present disclosure.

FIG. 22A through FIG. 22H are graphs showing performance of HarPE, DHF, and band filtration in non-invasive fetal SpO₂ estimation vs. ground-truth fetal SaO₂ readings, according to at least one embodiment of the present disclosure.

### DETAILED DESCRIPTION

### 1. Introduction

A method and apparatus are presented for separating quasi-periodic mixed-signals in the time-frequency space using only a single input data sample under the assumption that: (1) the signals are quasi-periodic and non-stationary; (2) only a single detector measurement is available; and (3) signal fundamental frequencies, but not their amplitudes, are known either through auxiliary sensing modalities or preliminary analysis of the mixed signal.

Notably, this method addresses two major challenges: (1) the potential overlap of signal frequencies, which renders classic frequency-based filtering techniques ineffective; and (2) the limitation of having only a single sample, which precludes the use of traditional machine learning methods that require a training dataset.

The present disclosure which is referred to herein as “Deep Harmonic Finesse” or “DHF”, separates signals in iterations (rounds). In each iteration, the problem is approached as a process of masking selected regions of the mixed signal's spectral representation, and in-painting them using a deep prior structure inspired by the Deep Prior approach. The masking step leverages the known frequency information of the source signals to conceal information of all but one of the signal sources. The process then in-paints the masked parts of the spectrogram to eliminate all masked signal sources from the mix and recover the target signal values. The recovered signal is removed from the mix, and the process is iteratively applied to the residual signal.

In at least one embodiment, the structural implicit priors can include two key components: a signal pattern-aligner and a specialized harmonic convolutional neural network. The pattern-aligner resamples and transforms the input signal into a new temporal space, providing the network with an input that aligns well with its expected deep prior pattern.

To validate the effectiveness of the present disclosure, the technique was applied on both synthesized data, and in-vivo data captured in animal studies using a wearable Transabdominal Fetal pulse Oximetery (TFO) device. This device senses a noisy Photoplethysmography (PPG) signal influenced by three quasi-periodic dynamics: respiration, maternal pulsation, and fetal pulsation. Compared to the best competing approach applied to the synthesized data, it was found that the method of the present disclosure improves signal-to-distortion ratio and mean squared error of extracted signals by an average of 26% and 80%, respectively. Compared to prior work applied to the in-vivo data, it was found that this new method improves the correlation between estimated fetal blood oxygen saturation and the ground truth by 80.5%.

### 2. Signal Separation with Limited Data

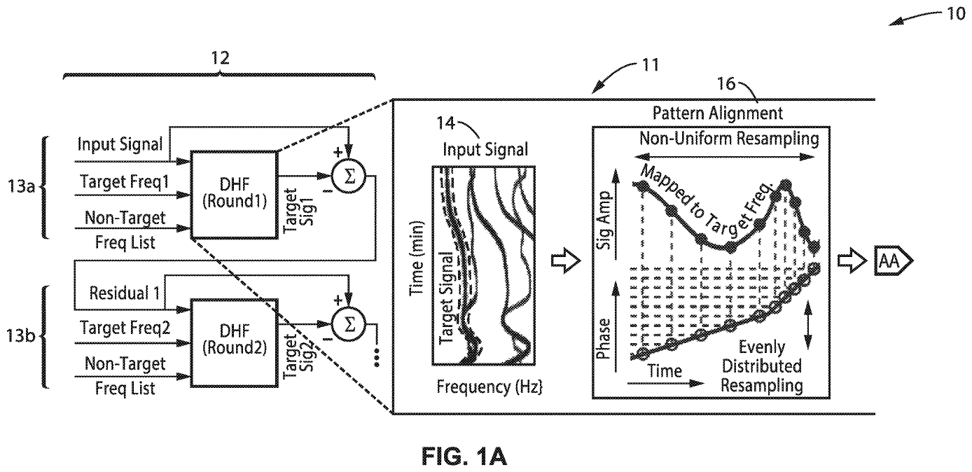

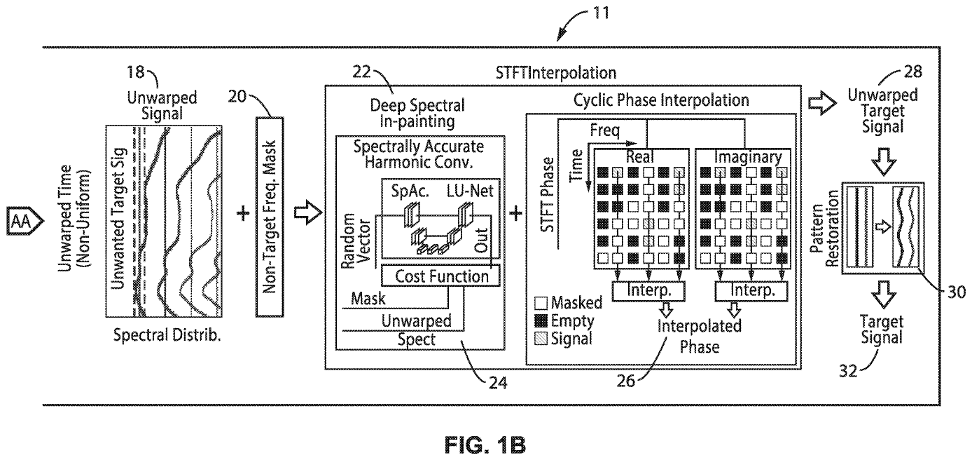

FIG. 1A and FIG. 1B illustrate an example embodiment 10 of the disclosed DHF method. The figure depicts a DHF processing block 11, and to the left of which is shown a diagram 12 showing how each DHF block 13a, 13b corresponds to one round of signal separation. DHF is an iterative method for signal separation in multi-source quasi-periodic signals using only a single mixed-signal in the dataset.

The first DHF iteration 13a is shown with Input Signal, Target Frequency, and Non-Target Frequency List supplied to the DHF iteration process. The DHF in round 1 generates a Target Signal 1 which is subtracted from the input signal at a summing junction, the output of which is Residual 1 which is used as the input in the next DHF iteration 13b, which produces Target Signal 2 output, which is subtracted from the Residual 1 input signal at a summing junction, and the iterations may continue as necessary in like manner.

Each round begins by selecting 14 the target signal to be separated, followed by pattern alignment 16 using resampling, then applying the unwarped signal 18 as seen in FIG. 1B, with a deep prior learning procedure with a non-target frequency mask 20, followed by a deep prior learning procedure 22 for in-painting and cyclic phase interpolation 26 with the Short-Time Fourier Transform (STFT). The mask conceals all significant spectral components of sources other than the selected one. In-painting interpolates the masked regions of the selected signal, targeting amplitude and phase separately, to recover values during spectral overlaps and crossover.

The structural prior used for spectrogram in-painting can include two complementary components: a signal pattern aligner 16 and a specialized convolutional neural network (U-Net) referred to herein as the Spectrally Accurate Light U-Net (SpAc LU-Net) 24. The pattern aligner 16 resamples and unwarps the mixed signal, transforming it into a temporal space where the selected source is strictly periodic (holding a steady fundamental frequency of 1 Hz). Given the strict periodic pattern over time and the harmonic patterns in spectrum, SpAc LU-Net defines the neighborhood in time and frequency by dilation and accessing integral multiples of a target bin, respectively. Concurrently, phase interpolation 26 is performed by separately interpolating the real and imaginary parts of each frequency bin's phase over time, and then recalculating the interpolated phase. The spectrogram in-painting results, joined with the interpolated phase information as an unwarped target 28, are then passed through the Inverse Short Time Fourier Transform (ISTFT) and transformed 30 back to the original steady-time space (referred to as pattern restoration) to deduce the separated signal time-series and produce target signal 32. After subtracting the separated source from the mixed signal, the residual is used for subsequent rounds of signal separation, as was seen between iterations 13a, and 13b.

It should be appreciated that in the original temporal space, the signal samples are equally spaced in time, meaning the sampling rate remains constant (steady-time space). The signals are then transformed by resampling into a new temporal space where the target signal becomes strictly periodic. In other words, the samples are no longer equally spaced in time, but instead the temporal samples are selected, such that the target signal component in the samples would be equally spaced in phase (steady-phase space). Both signals before and after the resampling step are a sequence of samples (e.g., x1, x2 . . . , etc. which refer to the sequence before resampling, and y1, y2 . . . , etc. refer to the sequence after resampling). For the X sequence, the temporal-distance between two consecutive samples is a fixed amount of time, i.e., classic sampling-based signal representation. The terms “steady-time”, and “original temporal space” are therefore equivalent. For the Y sequence, the temporal-distance between two consecutive samples is a fixed amount of time in a “new temporal space”. The consecutive samples also have the property of having a fixed amount of “phase difference” between target signal components of their corresponding points in the original temporal space, and thus the term “steady-phase” can be used. For example, let z1, z2, z3 be the points in the original X space that correspond to y1, y2, y3. The phase difference between target signal component of z1 and z2, is the same as the phase difference between target signal component of z2 and z3. It should be appreciated that z points are not necessarily the same as x points.

### 2.1. Target Pattern Alignment

In each round of signal separation, specific to a chosen target source, the disclosed method preprocess the mixed signal, X, and unwarps it with respect to the fundamental frequency of the target source, fₜₛ[n], locking a constant fundamental frequency, such as of 1 Hz, for that particular source in the unwarped signal, X′.

The original mixed signal space is defined in Equation 1. With a full period of the quasi-periodic target source showing a phase change from 0 to 2π, Equation 2 computes the unrolled phase at each sampling time, t[n].

$$( t [ n ] , X [ n ] ) , \qquad ( 1 )$$

$$where$$

$$t [ n ] = n Δ t = \frac{n}{{F}_{s}}$$

$$Φ [ n ] = 2 π {\sum }_{i = 1}^{n} {f}_{ts} [ i ] Δ t = \frac{2 π}{{F}_{s 0}} {\sum }_{i = 1}^{n} {f}_{ts} [ i ] \qquad ( 2 )$$

Fluctuations in the fundamental frequency result in a variable phase interval between successive samples. To unwarp to a constant fundamental frequency of 1 Hz, the new phase intervals between every pair of consecutive samples of X′ must remain constant, as indicated in Equation 3. The unwarped signal X′ is calculated by two sequential interpolations, detailed in Equations 4 and 5, first finding the timestamps of the evenly distributed phase intervals, t′[m], and then the mixed signal values at those timestamps, X′[m].

$$∀ m , {Φ}^{′} [ m ] - {Φ}^{′} [ m - 1 ] = \frac{2 π}{{{F}^{′}}_{s}} \qquad ( 3 )$$

$$∀ m , {Φ}^{′} [ m ] - {Φ}^{′} [ m - 1 ] = \frac{2 π}{{{F}^{′}}_{s}} \qquad ( 4 )$$

$$∀ m , {Φ}^{′} [ m ] - {Φ}^{′} [ m - 1 ] = \frac{2 π}{{{F}^{′}}_{s}} \qquad ( 5 )$$

### 2.2 Deep Prior Architecture

The pattern-aligned time-frequency spectrogram of quasi-periodic signals encapsulates critical features that need to be considered when constructing effective priors for the task of signal separation. These features outline the spatial neighborhood in both the time and spectral dimensions. Locking a constant fundamental frequency for the target source suggests that, to access preceding and subsequent amplitudes of that source over time, the convolutional kernel should exclusively examine the same frequency bin via dilation in time. Neighbors in frequency are defined as integral multiples of the current frequency, representing the harmonics, as opposed to the immediate next pixel (as in convolutional kernels).

Equation 6 below takes the following equation for the convolution operation between the noisy time-frequency spectrogram, X[ω,τ], and the kernel K with H total harmonics at any specific frequency location, \hat{ω}, and time, \hat{t}),

$$( X * K ) [ \hat{ω} , \hat{t} ] = {\sum }_{k = 1}^{H} {\sum }_{C = - T}^{T} x [ k \hat{ω} , \hat{t} - t ] K [ k , t ]$$

and extends it to add time dilation for constant frequency access in time:

$$( X * K ) [ \hat{ω} , \hat{t} ] = {\sum }_{k = 1}^{H} {\sum }_{C = - T}^{T} x [ k \hat{ω} , \hat{t} - {D}_{conv} t ] K [ k , t ] \qquad ( 6 )$$

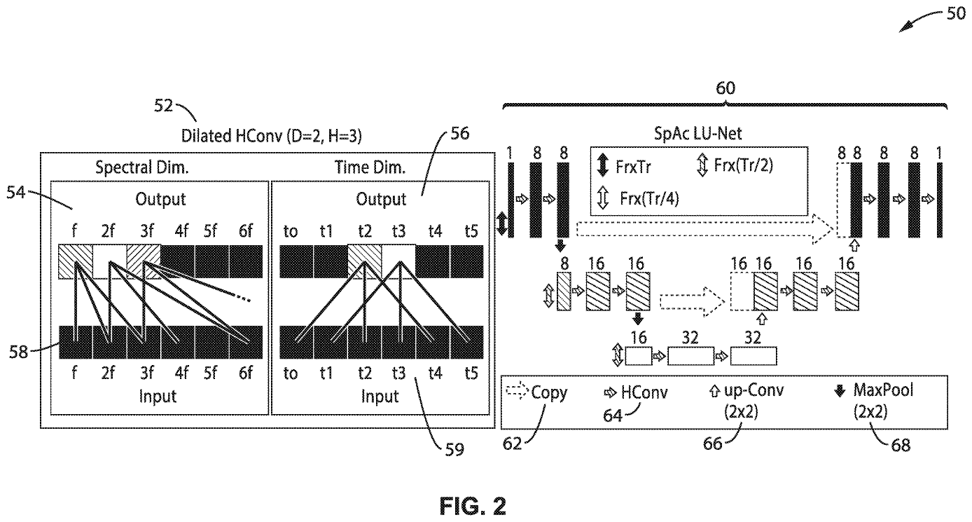

FIG. 2 illustrates an example embodiment 50 of the disclosed SpAc LU-NET structure 60 and the function of the dilated harmonic convolution 52 in frequency (spectral) 54 and time 56 dimensions. The outputs in the spectral and time dimensions are shown being mapped to inputs 58, 59.

At least one embodiment of the present disclosure has adapted this “U-Net” architecture as the foundation of the disclosed neural structure while substituting standard convolution kernels with dilated harmonic convolutions. Moreover, two design principles are maintained for harmonic integrity. Firstly, pooling in the frequency dimension is prohibited, ensuring the size of this dimension remains unchanged throughout the network. Secondly, only forward integral multiples are regarded as harmonic neighbors to guarantee spectral accuracy at all frequencies.

The disclosed SpAc LU-NET structure 60 is shown from upper left is shown processing FrxTr signals and performing Harmonic Convolutions (HConvolutions) 64, copy operations 62 along with MaxPool operation 68 to the center row of the figure as Frx(Tr/2), and to the bottom row of that figure as Frx(Tr/4), each of which also depicts H convolutions and copies them with up-conversions 66. In FrxTr the Fr is the frequency range representing the spectral dimension and Tr is the time range representing the time dimension. It should be appreciated that MaxPool (Max Pooling) is a type of operation in convolution neural networks that follows individual convolutional layers to reduce dimensionality by reducing the number of elements in the output from the previous convolutional layer.

SpAc LU-Net (Spectrally Accurate LU-Net) is a neural network model that is developed for in-painting of masked STFT spectrograms. SpAc LU-Net embeds into its architectural design prior information about the quasi-periodic signals that are to be separated. For example, in case of the physiological signals as studied herein, it defines the local neighborhood in time and frequency through dilation and integral multiples (harmonics) of a target frequency bin, respectively. During in-painting step of the method, SpAC LU-Net generates a STFT magnitude map, which is as similar as possible to a given masked STFT magnitude map. The similarity comparison is limited to the corresponding unmasked regions of the STFT maps.

### 2.3. Signal Separation by in-Painting

In at least one embodiment, masking and in-painting are utilized to separate sources in quasi-periodic mixed signals. During each round, a binary mask is utilized to conceal all significant harmonics of non-targeting sources from the cost function. The in-painting cost function is similar to that used for image in-painting applications. The selective visibility of the mask allows the optimizer to focus solely on the target source, in-painting the missing cross-overs and overlaps to retrieve the desired source.

Equation 7 presents the in-painting cost function, wherein S_{mixed} and Sₒᵤₜ represent the mixed spectrogram and the neural network's output spectrogram, respectively.

$$E ( {S}_{o u t} ; {S}_{mixed} ) = {‖ mask * ( {S}_{o u t} - {S}_{mixed} ) ‖}^{2} \qquad ( 7 )$$

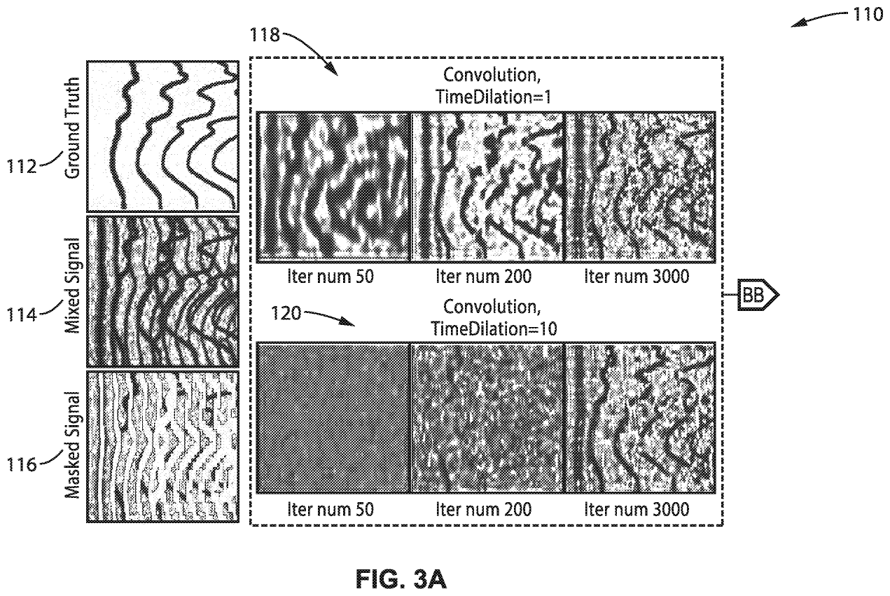

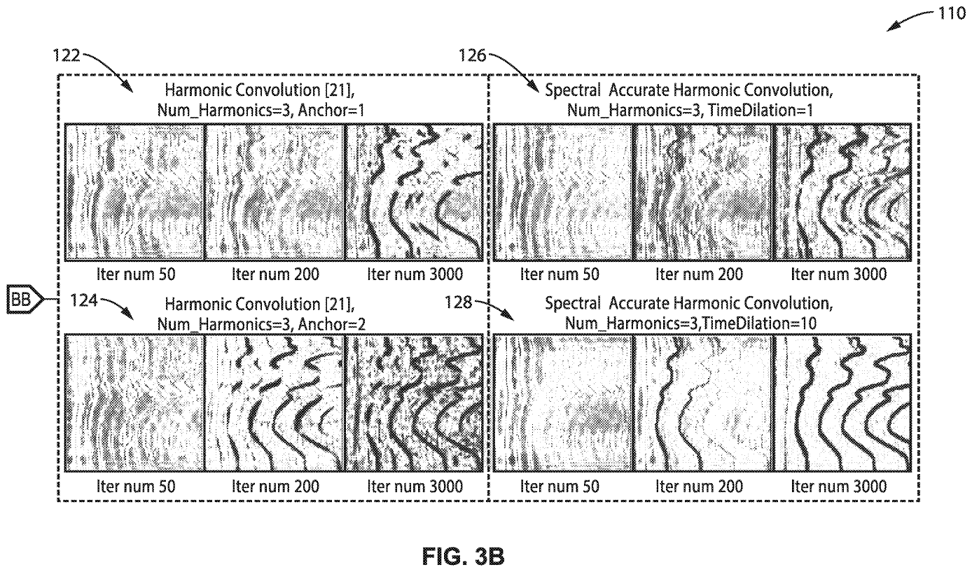

FIG. 3A and FIG. 3B illustrate an investigation 110 of the effectiveness of the disclosed neural network structure, SpAc LU-NET with harmonic convolutions, in its role as implicit priors for learning quasi-periodic time-frequency patterns. The figure depicts the ideal expected results on the left of the figure as Ground Truth 112 in comparison with the Mixed Signal 114 and Masked Signal 116. It should be appreciated that these figures, as well as many of the other figures in this disclosure were converted from high resolution color images to low resolution black and white images in accordance with patent office guidelines; whereby some of the details may be difficult to discern in the figures, despite the contrast enhancement performed on these images.

The remainder of FIG. 3A and FIG. 3B provides a comparative analysis of learning outcomes from different convolution kernels and network configurations receiving the same masked image. A comparison between conventional convolution kernels 118, 120 and harmonic convolution layers 122, 124 reveals the repetitive vertical frequency patterns, which are noticeable even at the initial stages of learning. However, the baseline harmonic convolution setup uses anchors larger than one, which permits inaccurate backward harmonic neighboring, and max-pooling in frequency, leading to inaccurate spectral representation. This setup weakens the prior structure and increases the noise.

The Spectrally Accurate design 126, 128, builds on the previous setup by maintaining a fixed anchor, in this disclose fixed at 1, and eliminating frequency folding, and shows a notable reduction of noise especially when using the time dilation that aligns well with the constant-frequency patterns after the unwarping step.

### 2.4. Cyclic Phase Interpolation

Phase interpolation of the STFT data is conducted independently from the spectrogram in-painting. Initially, phase information is extracted for each signal separation round from the unwarped STFT complex values. Utilizing the previously generated mask, the phase is then estimated during overlaps and crossovers through interpolation. Owing to the strictly periodic patterns in time-frequency space achieved after pattern alignment, each frequency bin is interpolated over time individually, yet concurrently with others. For each bin, both the real and imaginary components of the phase are interpolated, with subsequent determinations of interpolated phase by integrating these results. This is to ensure the cyclic nature of the phase is preserved during interpolation.

### 3. Experimental Results

### 3.1. Synthesized Data

To expedite testing for the present disclosure we have created a tool for generating synthesized quasi-periodic time series, as characterized by the desired input function per period, time duration per period list, and amplitude per period list.

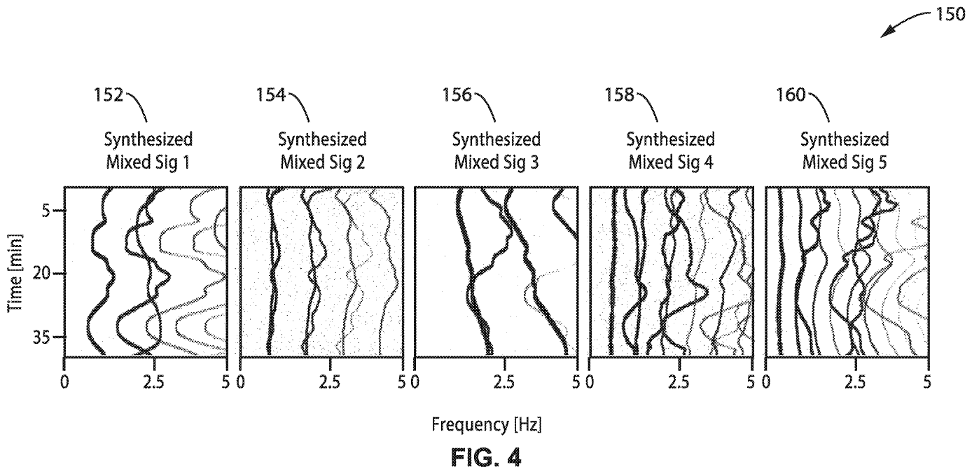

FIG. 4 illustrates examples 150 of spectrograms of generated mixed signals. Specifically, in this test sequence five distinct synthesized mixed quasi-periodic signals (MSig1 152, MSig2 154, MSig3 156, MSig4 158, MSig5 160) have been generated, with sampling frequency of 100 for TFO application. Each mixed signal has 2-3 sources for respiration, maternal pulsation, and fetal pulsation. The respiration PPG shape is extracted from real sheep experiments after filtering out other dynamics. The pulsation PPG shape was randomly extracted from MIMIC-IV dataset. The details of the synthesized signals are further explained in Table 1.

Mixed Signals 1-3 (152, 154, 156) each have two sources (maternal pulsation and fetal pulsation). The sources in Mixed Signal 1 152 shows interference in the second harmonic of the target source, while Mixed Signal 2 154 has interference on the first harmonic. In Mixed Signal 3 156 the second source has less than ×0.1 of the dominant source's amplitude. Mixed Signals 4-5 158, 160 have three signals each (respiration, maternal pulsation, and fetal pulsation), with differences in overlap duration and complexity, and less than ×0.1 of amplitude on their third source compared to their first source.

### 3.2. Signal Separation Results: Synthesized Data

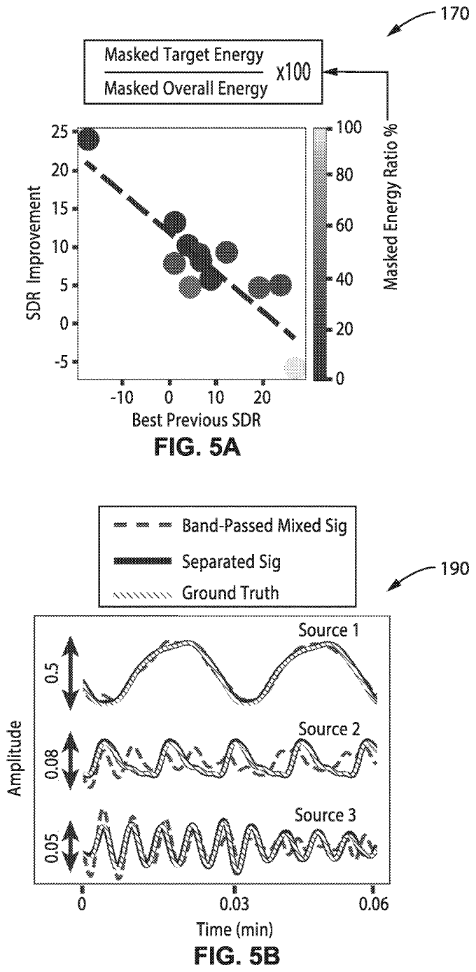

FIG. 5A and FIG. 5B illustrate example results 170, 190 of SDR comparisons and synthesized signal separation when DHF is applied to mixed Signal 5, as was described for FIG. 4. The waveform shows a very close decomposition for all three sources. Statistics are further explored of DHF performance in comparison to previous signal separation methods when applied on the generated mixed signals.

Metrics: Signal to Distortion Ratio (SDR) and Mean Squared Error (MSE) are presented for each separated source from the five input mixed signals. For averaging MSE values, geometric averaging is employed, whereas for SDR averaging, arithmetic averaging is utilized in the original linear scale.

Experiment Setup: The spectrograms are generated with window and stride of 60 s and 15 s, respectively. It has been found in our study that an increased time dilation parameter can improve the performance for extracting sources with longer masked sections. Commonly, the masked sections are longer when targeting sources with higher fundamental frequency than other sources in the mix. This is expected since the stride between harmonics of low-frequency sources are smaller, and therefore, the masked area on the spectrogram is larger. A dilation was employed of either 13 or 15, making the decision on a case-by-case basis according to the specific masking situation.

Certain conventional signal separation mechanisms, such as Independent Component Analysis (ICA) and Principal Component Analysis (PCA), do not accommodate single-detector signal separation. As a result, these were excluded from a comparison study. The results from the present disclosure are compared against six previous signal separation methods, EMD, VMD, NMF, REPET and REPET-Extended, and spectral masking as presented in Table 2. These results are all calculated on band-pass filtered mixed signals (MSig) between [0 Hz, 12 Hz].

Discussion: The results in Table 2 show that the disclosed DHF method achieves an approximate 26% (2.3 db) improvement in Signal-to-Distortion Ratio (SDR) and 80% reduction in Mean Squared Error (MSE) on average, compared to the best previous signal separation algorithms. This improvement is particularly true for sources with less than ×0.1 amplitude of the dominant source (such as mixed signal 3 source 2, mixed signal 4 source 3, and mixed signal 5 source 3), wherein DHF demonstrates remarkable effectiveness. For these three low-power scenarios, the disclosed method shows an average SDR improvement of 7.2 db and a 92% average MSE improvement over the best prior methods.

In FIG. 5A a further analysis indicates that the SDR improvements achieved with DHF compared to the highest SDRs of previous methods. The findings indicate that DHF fills a critical gap in existing methods, particularly excelling in scenarios where others falter. A heatmap is employed to illustrate the relationship between the Masked Energy Ratio, defined as the percentage of masked target energy to overall masked energy per signal separation round, and DHF's superior performance. Previous methods tend to struggle with low masked energy ratios, attempting to isolate low-energy signals from strong overlapping interference, a challenge where DHF notably outperforms its predecessors.

In FIG. 5B is shown an example of a separated waveform after three rounds of DHF were applied on a mixed signal with three sources: Source 1, Source 2, and Source 3. It can be seen in the figure that the separated Source 1, Source 2, and Source 3 signals closely map to the ground truth Source 1, Source 2, and Source 3 signals showing improved separation performance compared to the band-passed signal around each source.

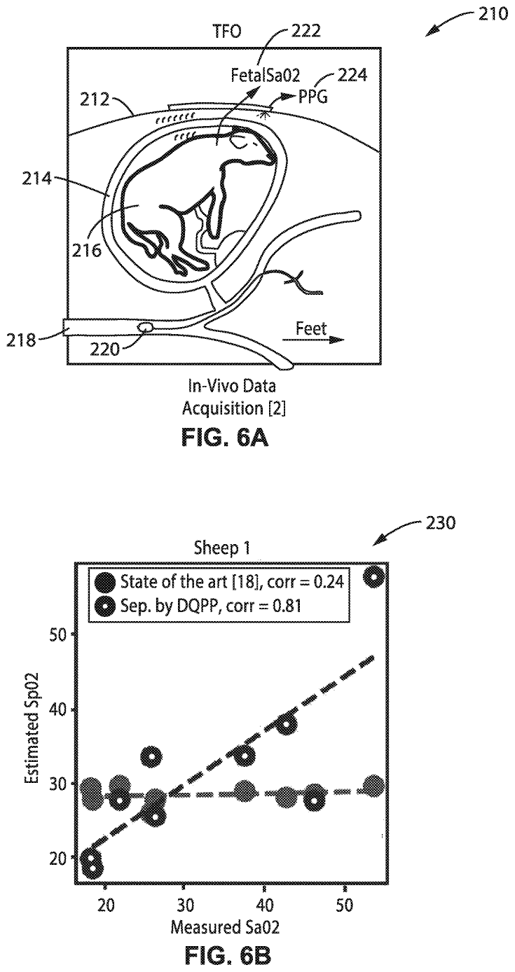

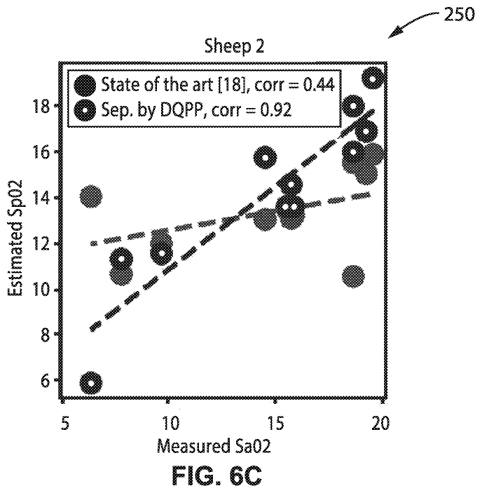

3.3. Signal Separation Results: in-vivo SpO₂ Estimation FIG. 6A through FIG. 6C illustrate example data acquisition 210 and Sa02 results 230, 250. In the following discussion, it should be appreciated that Sp02 is oxygen saturation as measured by the pulse oximeter, while Sa02 is oxygen saturation as measured by the blood gas analysis.

The performance of this aspect of the present disclosure on fetal SpO₂ estimation in an in-vivo dataset of TFO from two pregnant ewes. This dataset consists of 40 minutes of continuous mixed PPG signals at 740 nm and 850 nm wavelengths gathered from pregnant ewe's abdomen and ground-truth fetal blood oxygen saturation readings, i.e., SaO₂, measured from blood-draws with the time distance of 2.5, 5, and 10 minutes.

In FIG. 6A the TFO data acquisition method used for the adopted dataset is depicted. FIG. 6A shows abdomen 212 of an ewe with uterus 214, and ewe fetus 216, with a pulse oximeter 220 inserted into the Aorta of the ewe. Since the ground-truth fetal PPG signal 224 is not accessible, the correlation of SpO₂ estimation with measured SaO₂ readings is reported when the fetal signal is separated using spectral masking and DHF method. Comparisons are performed against spectral masking since it has shown improved performance in relation to previous testing.

SpO₂ Estimation: in one embodiment the described method for non-invasive estimation of fetal blood oxygen saturation uses pulse oximetry (SpO₂). Equation 8 presents the linear regression method for SpO₂ estimation where Y, k, and R are SaO₂ measurement vector, a constant regularizing term, and the modulation ratio vector, respectively; and w₀ and w₁ are the learned parameters. Equation 9 explains the modulation ratio calculation method which is a classic parameter in pulse oximetry. AC and DC represent the dynamic and static portions of the PPG signal at each wavelength. To aid the comparisons, in this work R values are averaged over a 2.5 minute window centered around each blood draw time stamp as was used in previous work.

$${Y}^{′} = \frac{1}{Y + k} = {w}_{0} + {w}_{1} R , \qquad ( 8 )$$

$$k = 1.885$$

$$R = \frac{{( AC / DC )}^{{λ}_{1}}}{{( AC / DC )}^{{λ}_{2}}} \qquad ( 9 )$$

Discussion: FIG. 6B and FIG. 6C present the SpO₂ estimation performance for both sheep, Sheep1 230 and Sheep2 250, depict comparing the results when the separated fetal signal is obtained through spectral masking and DHF. Comparing the correlation between SpO₂ estimations and SaO₂ readings, the disclosed method improves the correlation from 0.24 to 0.81 and from 0.44 to 0.92 in sheep 1 and sheep 2, respectively (80.5% average error improvement from ideal correlation of 1 between SpO₂ estimations and SaO₂ readings).

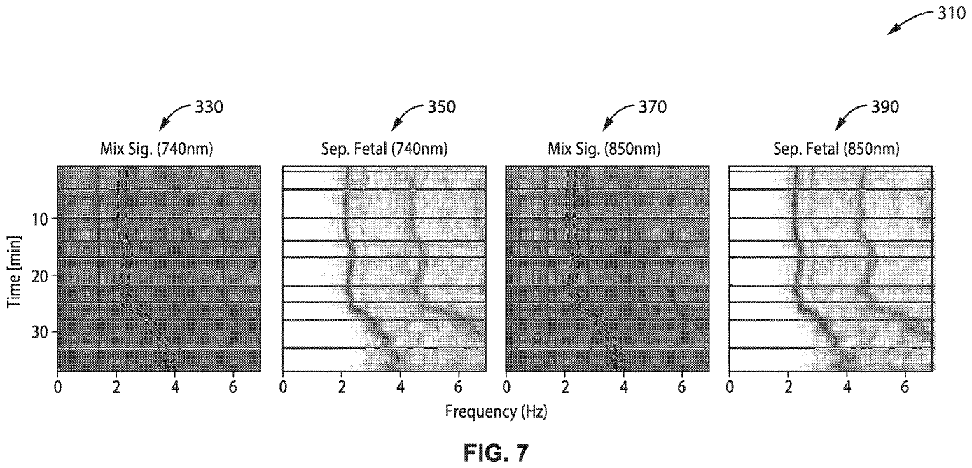

FIG. 7 illustrates example results 310 for measured mixed signal spectrograms 330, 370 for sheep 2 at 740 nm and 850 nm and the separated fetal signal 350, 390 at each wavelength using the DHF method. The horizontal lines in these figure mark the blood draw timestamps and the dashed line box highlights the lamb's fetal signal before signal separation is performed.

### 4. DHF Summary

Limitations in dataset capacity and quantity, present in many quasi-periodic signal separation applications in wearable systems, have hindered their success despite their significant potential. Incorporation of application-specific prior knowledge can enhance signal separation performance with limited data. In this work, source frequencies, such as captured through additional sensors or posterior analytical methods, are utilized to first mask the undesired frequencies for a specific target source, and second to transform the time-frequency representation to create a stronger alignment between the target source and implicit deep priors built into the structure of the disclosed neural network. The present disclosure has showcased a Transabdominal Fetal pulse Oximetery (TFO) application, using both synthesized and in-vivo data, where it shows significant improvement compared to the state of the art.

### 5. DHF Extended

### 5.1. Introduction

Wearable biosensor technologies are essential to advancing personalized healthcare by enabling continuous monitoring of physiological signals, enabling timely interventions and enhanced patient outcomes. The sensed signals by such devices are typically influenced by multiple quasi-periodic physiological phenomena, such as heartbeat, respiration, and Mayer waves, which may overshadow the signal of interest. Examples include tissue sensing and blood glucose monitoring applications in which, the target signal carries much less energy compared to the dominant in-band interfering sources, undermining conventional filtration and signal separation methods. Moreover, the prohibitive cost and practical challenges of collecting extensive and comprehensive datasets make straight-forward deep learning techniques less effective. Consequently, there is an unmet need for signal separation algorithms that can work effectively with fewer sensors, and can leverage small, unlabeled or partially labeled datasets.

Prior-based methods are commonly used to enhance learning generalizability when data is limited and under-representative. Integrating statistical priors directly into a cost function, the traditional approach, encounters challenges in complex systems with real-world data, mainly due to intricate or impractical modeling requirements. An alternative is encoding priors within the neural structure, which offers model flexibility while inherently increasing bias. In advancing this concept, it was notably demonstrated that implicit priors embedded within the structure of a deep generative network can perform image restoration effectively, requiring only a single image and no additional training data.

In Deep Harmonic Finesse (DHF) separating single-channel quasi-periodic mixed signals are separated without additional training data. DHF leverages the harmonic structure in the time-frequency domain to mask interference and restore non-stationary amplitude over time, assuming knowledge of fundamental frequencies of signal sources that can be acquired through either frequency inference algorithms or auxiliary sensing. DHF consists of two closely interconnected components: the adaptive pattern alignment unit and the complementary deep prior structure. To isolate a non-stationary quasi-periodic target signal from the mixed input, the pattern alignment unit resamples the mixed signal according to the varying target frequency, ensuring a strictly periodic pattern in the resampled time-frequency space. This aligned pattern is then processed by the specifically designed deep prior neural structure, which efficiently separates the target signal. Stated differently, the process of signal separation is approached as a task of masking non-target signals in the time-frequency space, and inpainting areas of overlap with the target signal. A separate unit interpolates the phase data for an accurate reconstruction of the target signal in the time domain.

The present disclosure provides a number of contributions, including the following. Providing a novel deep learning-based method to separate single-channel, mixed quasi-periodic signals with known frequencies, but unknown phase and amplitude, when only a small amount of data is available. The method functions during prolonged overlap and crossover of constituting signal frequencies, such as strong in-band noise, which render frequency-based filtering techniques ineffective.

The described method, dubbed DHF, addresses an inherent limitation of classic statistical signal separation techniques, whose performance degrades when the faint and noisy target signal is frequently overshadowed by dominant interfering signals with significantly higher energy. Furthermore, it addresses a critical gap in machine learning-based approaches to signal separation whose application is limited when a substantial volume of training data is unavailable.

The disclosure demonstrates the effectiveness of this approach using both synthesized data, and in-vivo data captured in animal studies. Compared to the best competing algorithm applied to the synthesized data, the disclosed method improves signal-to-distortion ratio and mean squared error of extracted signals by an average of 106% and 85%, respectively. Compared to the prior work applied to the in-vivo transabdominal fetal oximetry data, with the disclosed method improving the correlation between estimated fetal blood oxygen saturation and the ground truth by 80.5%.

### 5.2. Motivating Example

Transabdominal Fetal Pulse Oximetry (TFO) is a primary method under research for continuous and non-invasive monitoring of fetal oxygenation, which is a key determinant in assessing fetal health. Continuous monitoring of fetal oxygenation is essential to detect signs of fetal distress or asphyxia early, enabling more timely interventions compared to current intermittent and indirect methods. The non-invasive monitoring of fetal blood oxygen saturation using TFO reduces the risks associated with invasive procedures, such as potential infections or harm to the fetus or mother. Moreover, the method commonly uses light with red and infra-red wavelengths that are considered safe for the skin and body.

In one study TFO in-vivo data from pregnant ewes is collected with a specially designed non-invasive pulse oximeter that is placed on the ewe's abdomen. Then, fetal oxygen supply is controlled by inflating a balloon in maternal aorta, while collecting Photoplethysmography (PPG) signals at two wavelengths (740 nm and 850 nm) and ground truth blood oxygen saturation using blood draws from fetus at 3-5 minutes intervals.

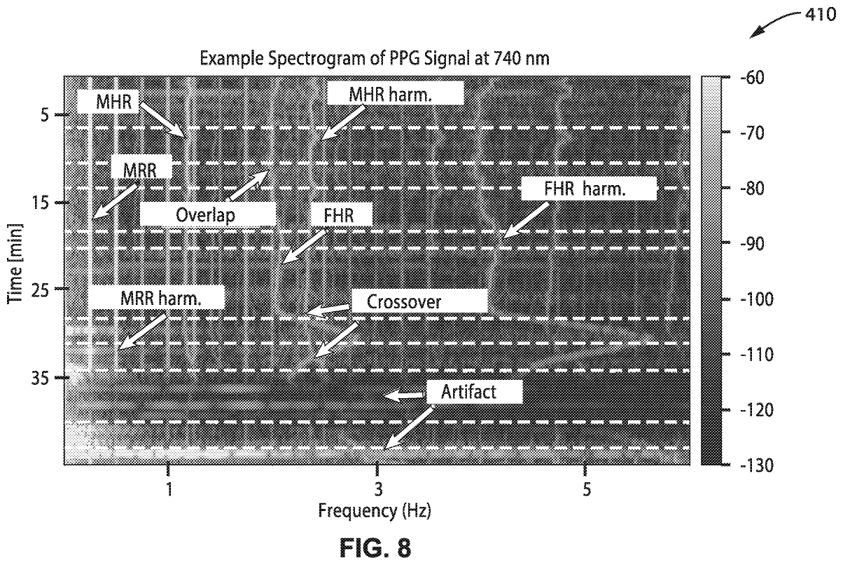

FIG. 8 illustrates 410 an example spectrogram in an in-vivo sheep study of TFO PPG reading using the aforementioned device. The mixed signals include three major quasi-periodic signals, maternal respiration, maternal pulse, and fetal pulse. The fundamental frequencies of these signals are labeled as Maternal Respiratory Rate (MRR), Maternal Heart Rate (MHR), and Fetal Heart Rate (FHR). This study determines that while single-body pulse oximetry is a routine standard of care, multi-body pulse oximetry in a TFO application is significantly more challenging due to multiple reasons. (a) The need for light to penetrate multiple tissue layers to reach the fetus diminishes the signal strength. Consequently, the noise level received might be comparable to the desired fetal heart-beat signal, complicating accurate detection. (2) Moreover, the dominant maternal signal can overshadow the weaker fetal signal. This problem is exacerbated in sheep studies, where the maternal respiration dynamic is much stronger than both the maternal and fetal dynamics, overshadowing and complicating their detection. (3) PPG sensors are also highly sensitive to motion artifacts, which are inevitable in the pre-birth environment. Therefore, the effective separation of fetal signal from collected PPG signals is complicated.

The substantial noise in the collected PPG signals makes existing filtration or signal separation approaches insufficient for accurate isolation of the fetal signal and reliable oxygen saturation estimation. The present disclosure demonstrates the superior performance of DHF signal separation in improving fetal oxygen saturation estimation versus previous state-of-the-art signal isolation methods. Building upon this study, this section of the disclosure also generates a synthesized TFO dataset to evaluate the effectiveness of the DHF signal separation method with access to the ground-truth separated signals, an ability not available with actual TFO data.

### 5.3. Related Work

Single-detector signal separation methods can be broadly classified into three categories: (1) Analytical component decomposition methods with or without consideration of the periodic behavior, (2) Deep learning methods that require ample datasets, and (3) Deep prior learning methods.

### 5.3.1 Analytical Methods

Empirical Mode Decomposition (EMD) and Variational Mode Decomposition (VMD), as well as their extension methods, decompose a time-domain signal into Intrinsic Mode Functions (IMFs), each representing different sources.

Empirical Mode Decomposition (EMD) is citation [1]: Norden E Huang, Zheng Shen, Steven R Long, Manli C Wu, Hsing H Shih, Quanan Zheng, Nai-Chyuan Yen, Chi Chao Tung, and Henry H Liu. 1998.

Variational Mode Decomposition (VMD) is citation [2]: Konstantin Dragomiretskiy and Dominique Zosso. 2013. Variational mode decomposition. IEEE transactions on signal processing 62, 3 (2013), 531-544. The empirical mode decomposition and the Hilbert spectrum for nonlinear and non-stationary time series analysis. Proceedings of the Royal Society of London. Series A: mathematical, physical and engineering sciences 454, 1971 (1998), 903-995.

EMD considers a signal's local extremas for extracting IMFs while VMD defines a variational problem assuming each source to have a compact frequency representation around a central frequency, which tends to provide better frequency separation than EMD. Non-negative Matrix Factorization (NMF) operates in both time and frequency domains. In time-frequency, it breaks down the 2-D matrix into two product matrices, which can be interpreted as distinct sources.

Presented for the separation of sound signals in the time-frequency domain, the REpeating Pattern Extraction Technique (REPET) works under the assumption that a sound signal can be represented as a sum of a repeating background with a varying foreground. An extension of REPET, REPET-Extended, can adapt to changes in the repeating structure over time, providing better separation quality when the repeating parts of the signal are non-stationary.

REpeating Pattern Extraction Technique (REPET) & REPET-Extended is citation [4]: Zafar Rafiiand Bryan Pardo.2012. Repeating pattern extraction technique (REPET): A simple method for music/voice separation. IEEE transactions on audio, speech, and language processing 21, 1 (2012), 73-84.

### 5.3.2. Data-Intensive Learning Methods

Leveraging the availability of extensive datasets in the audio domain, extensive research has been conducted on audio source separation using both supervised and unsupervised methods. Applied in the time-frequency domain, data intensive learning methods employed deep clustering to learn embeddings for each spectrogram bin. These embeddings are then utilized to derive an optimal mask for separating sources in single-channel multi-speaker audio scenarios. Building on this, enhanced music source separation was performed by integrating the deep clustering method with traditional supervised learning. That approach employed a dual-headed network that simultaneously performed clustering and ideal mask estimation. Following the success of U-Net neural architecture in biomedical image segmentation, the U-Net structure was adopted in supervised audio source separation. The U-Net's multi-scale structure effectively captures both high-level features and fine details, facilitating the learning of ideal masks for singing voice separation in the time-frequency domain. Addressing the inherent limitations of time-frequency domain separation, such as phase reconstruction challenges, task-specific fine-tuning of STFT parameters, and latency issues with high-resolution STFT, one technique modifies the U-Net architecture for one-dimensional time-series (audio wave) input. Further innovating in this area, are signal separation methods in time domain with a focus on real-time speech applications. They introduce two network variations, one leveraging Long Short-Term Memory (LSTM) units (neural networks) and the other convolutional layers, which are adept at learning representations of separated audio signals from mixed speech inputs. The output representations are then converted back into waveform format using a linear decoder. One approach here focuses on enhancing high-frequency accuracy of separated signals by reconstructing high-frequency components by comparing input mixed spectrograms and separated signal spectrograms.

To address the limitations of supervised training methods that rely on synthesized data with unrealistic/limited mixing models, conventional approaches have explored unsupervised methods to achieve higher generalizability without access to isolated sources. One proposal includes an unsupervised generative adversarial network method that models the distribution of individual sources within mixed signals. An unsupervised approach that separates signals with unknown number of sources has been used. The training examples are sum of two real-world single-channel mixed data, and the model is trained to find underlying sources by comparing their selective sum with the original mixed data. Taking this further, another approach used an innovative unsupervised framework for audio-visual on-screen sound separation, capable of isolating on-screen sounds. This approach does not restrict the sound domain to specific classes, accommodating variable numbers of sources, and eliminating the need for labels or visual segmentation. All these methods rely on the availability of large datasets, a condition which is not applicable to a large portion of real-world applications where excessive data acquisition is costly and impractical.

### 5.3.3. Deep Priors

In one conventional approach, it has been demonstrated that the structure of a generative convolutional deep neural network, f_{θ}, can be used as a prior for image denoising, in-painting and super-resolution tasks using only one sample in the dataset. The network receives a random vector, z, as input, and is subsequently trained to generate an output that is similar to a single, given noisy, low-quality or masked image, x₀. Therefore, without requiring a large dataset to train the network on, the training method minimizes the distance between the neural network's output and the input image. More formally, the training minimizes the cost function min_{θ}E(f_{θ}(z);x₀), where E is a task specific term. The cost function of the in-painting task, which has also been used in this work, is presented in Equation 10. The mask contains binary values, either 0 or 1, with a shape similar to the input image that conceals certain parts of the image from the cost function.

$$E ( x ; {x}_{0} ) = {‖ ( x - {x}_{0} ) \text{?} mask ‖}^{2} \qquad ( 10 )$$

In yet another conventional approach, audio can be processed by designing harmonic convolutions to show how time-frequency priors can emerge from such neural networks. The natural statistics of images and audio signals are different than images, and therefore, this particular prior design is allegedly different. Patterns of harmonics were used in the time-frequency plane to redefine neighborhood in frequency of the conventional deformable harmonic convolution. The convolution output at each frequency is the weighted sum of input at integer multiples of that specific frequency. This definition can be further extended to consider backward harmonic access by introduction of an anchor that multiplies integral multiples to anchor⁻¹.

Employing a deep convolutional prior technique, another conventional approach employed the auditory coherence and discontinuity feature of sound sources to separate the source signals using only a single-detector sound in time-frequency space. The technique utilizes two separate parallel networks per source that generate the signal and its silencer mask, mixing them together at their output to compare against the mixed input signal. However, this method is ineffective for separation of continuous quasi-periodic signals that inherently do not have silence gaps.

### 5.4. Improved Methodology

DHF provides a method of signal separation in multi-source quasi-periodic signals using only a single mixed-signal in the dataset. Each round begins by selecting the signal to be separated, followed by an improved deep prior learning procedure for masking and in-painting the Short-Time Fourier Transform (STFT). The mask conceals all significant spectral components of sources other than the selected one. In-painting interpolates the masked regions of the selected signal, targeting amplitude and phase separately, to recover values during spectral overlaps and crossover. The structural prior used for spectrogram in-painting can include two complementary components: a signal pattern aligner and a specialized convolutional neural network that we call the Spectrally Accurate Light U-Net (SpAc LU-Net). The pattern aligner resamples and unwarps the mixed signal, transforming it into a steady-phase space where the selected source is strictly periodic (holding a steady fundamental frequency of 1 Hz). Given the strict periodic pattern over time and the harmonic patterns in spectrum, SpAc LU-Net defines the neighborhood in time and frequency by dilation and accessing integral multiples of 1 hz distance from a target bin, respectively. Concurrently, phase interpolation is performed by separately interpolating the real and imaginary parts of each frequency bin's phase over time, and then recalculating the interpolated phase. The spectrogram in-painting results, joined with the interpolated phase information, are then passed through the inverse short time Fourier transform (ISTFT) and transformed back to the original space (referred to as pattern restoration) to deduce the separated signal time-series. After subtracting the separated source from the mixed signal, the residual is used for subsequent rounds of signal separation.

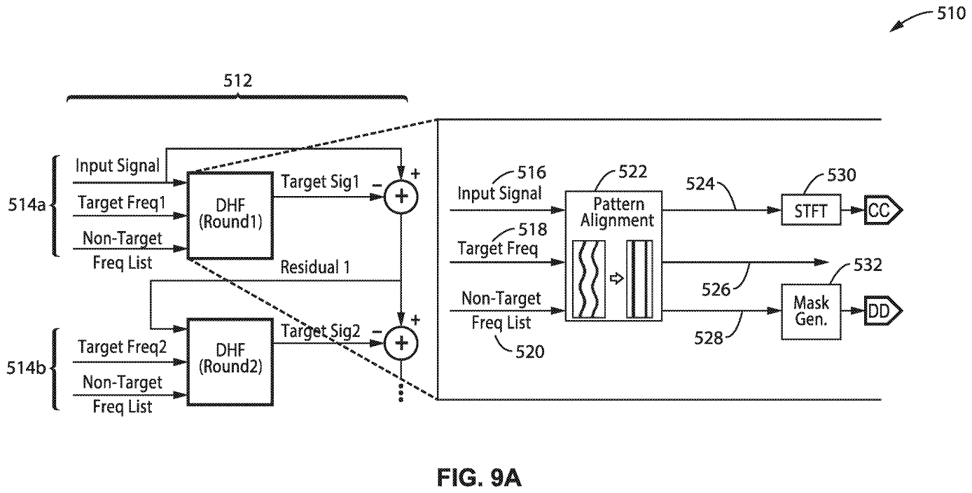

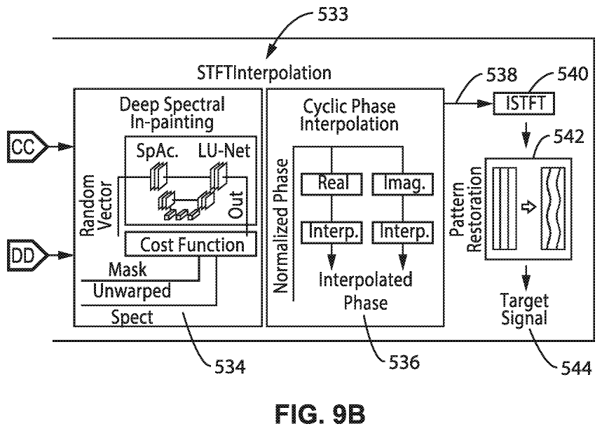

FIG. 9A and FIG. 9B illustrate an example embodiment 510 of the disclosed method, where each DHF block corresponds to one round of signal separation. This figure is quite similar to that seen in FIG. 1A and FIG. 1B, showing an iterative DHF process 512 which, by way of example, may have any desired number of rounds, herein exemplified as 514a, 514b.

In this figure the results from the residual from the first iteration is used as an input signal for the second iteration, and separated target signal in each iteration is subtracted from the input signal. Inputs to the DHF are shown with input signal 516, target frequency 518, and a non-target frequency list 520 to a pattern alignment process 522. From the pattern alignment process there are unwarped signals 524 passed to the STFT 530, strictly periodic target frequency 526, and unwarped non-target signals from the frequency list 528 to a mask generator 532. These signals are received for STFT interpolation 533 which includes deep spectral in-painting 534 and cyclic phase interpolation 536. Output 538 from this is to an inverse STFT (ISTFT) 540 to a pattern restoration process 542 from which the target signal 544 is output.

### 5.4.1. Target Pattern Alignment

The sensed mixed signal, X, is an additive combination of multiple quasi-periodic source signals Xᵢ, with non-stationary amplitude and fundamental frequency, aᵢ and fᵢ, respectively. Equation 11 captures the mixing model:

$$X [ n ] = {\sum }_{i} {a}_{i} [ n ] {X}_{i} ( {f}_{i} [ n ] , n ) + noise \qquad ( 11 )$$

In each round of signal separation, specific to a chosen target source, the disclosed method resamples the mixed signal and unwarps it with respect to the fundamental frequency of the target source, fₜₛ[n], locking a constant fundamental frequency of 1 Hz (in this embodiment) for that particular source in the unwarped signal, X′. In other words, the adaptively resampled signal patterns a strictly periodic target signal with constant frequency, and a varying amplitude over time. Therefore, the rate of samples in time is no longer uniform and varies based on original target frequency pattern. Instead, the samples will be evenly distributed in phase, for that reason, from this point forward the resampled space is termed the steady-phase space, in contrast to the original steady-time space.

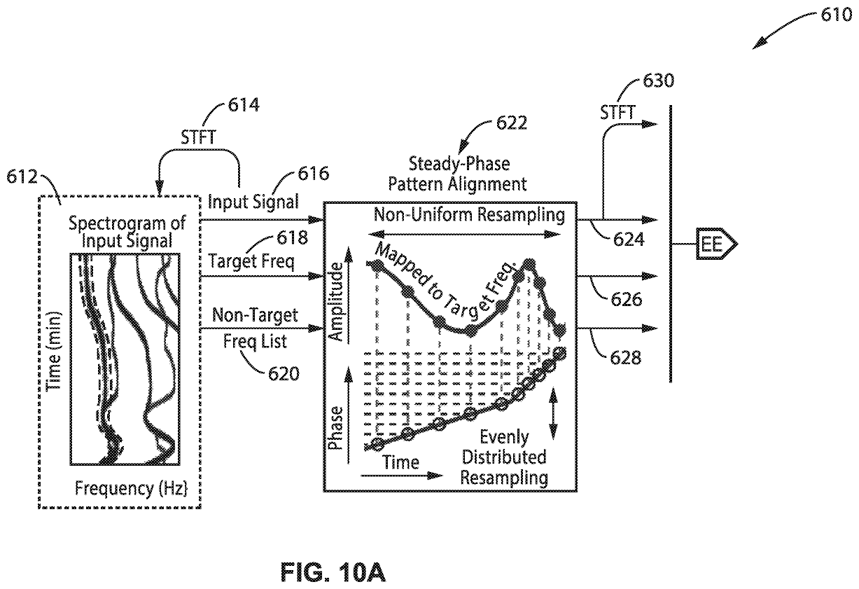

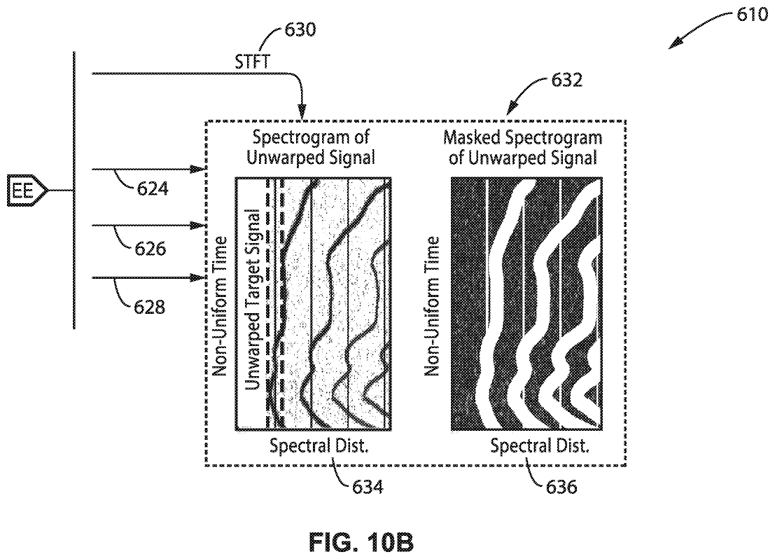

FIG. 10A and FIG. 10B illustrate an example embodiment 610 of the resampling approach and the spectrogram view of an example signal before and after pattern alignment. The pattern alignment unit also provides the spectral pattern of non-target sources in the steady-phase space, which is used for masking those signals during spectrogram in-painting and phase interpolation. The figure depicts the spectrogram 612 of the input signal, with STFT 614, and passing of signals to the Steady-Phase Pattern Alignment (SPPA) process 622. The received signals are the input signal 616, target frequency 618 and non-target frequency list 620. Block 620 represents some aspects of this process of non-uniform resampling in amplitude, and evenly distributed resampling in the phase domain. The SPPA process outputs an unwarped signal 624, periodic target frequency signal 626, unwarped non-target frequency list 628 and STFT 630 to block 632 shown representing a spectrogram of unwarped signals 634, and a masked spectrogram of unwarped signals 632.

The steady-time space (before resampling) is defined in Equation 12. With a full period of the quasi-periodic target source showing a phase change from 0 to 2π, Equation 13 computes the unrolled phase at each evenly spaced sampling time, t[n].

$$( t [ n ] , X [ n ] ) , \qquad ( 12 )$$

$$where$$

$$t [ n ] = n Δ t = \frac{n}{{F}_{s}}$$

$$Φ [ n ] = 2 π {\sum }_{i = 1}^{n} {f}_{ts} [ i ] Δ t = \frac{2 π}{{F}_{s 0}} {\sum }_{i = 1}^{n} {f}_{ts} [ i ] \qquad ( 13 )$$

Fluctuations in the fundamental frequency result in a variable phase interval between successive samples. To unwarp to a constant fundamental frequency of 1 Hz and transform the signal to the steady-phase space, the phase distance between every pair of consecutive samples of X′ must remain constant, as indicated in Equation 14. The steady-phase signal X′ is calculated by two sequential interpolations, detailed in Equations 15-16, first finding the timestamps of the evenly distributed phase intervals, t′^{[m]}, and then the mixed signal values at those timestamps, x′^{[m]}.

$$∀ m , {{Φ}^{′}}^{[ m ]} - {{Φ}^{′}}^{[ m - 1 ]} = \frac{2 π}{{{F}^{′}}_{s}} \qquad ( 14 )$$

$$( Φ [ n ] , t [ n ] ) \to ( {{Φ}^{′}}^{[ m ]} , {{t}^{′}}^{[ m ]} ) \qquad ( 15 )$$

$$( t [ n ] , X [ n ] ) \to ( {t}^{′} [ m ] , {X}^{′} [ m ] ) \qquad ( 16 )$$

To effectively filter out non-target signals within the steady-phase space, it is also necessary to compute the transformed frequencies of any interfering sources. This involves resampling performed similar to Equation 16, while using the ratio of the non-target frequency to the target frequency time-series, for each interfering source, as the input signal.

The steady-phased signals can be transformed back to the steady-time space by interpolation from the resampled timestamps, t′[m], to the original timestamps as outlined in Equation 17.

$$( {t}^{′} [ m ] , {X}^{′} [ m ] ) \to ( t [ n ] , X [ n ] ) \qquad ( 17 )$$

### 5.4.2. Deep Prior Architecture

The steady-phase spectrogram of quasi-periodic signals encapsulates critical features which are considered in deep neural priors structure. These features outline the spatial neighborhood in both the time and spectral dimensions. Locking a constant fundamental frequency for the target source suggests that, to access preceding and subsequent amplitudes of that source over time, the convolutional kernel can exclusively examine the same frequency bin via dilation in time, Dₜ. Neighbors in frequency are then defined as integral multiples of 1 Hz distance from the current frequency, D_{f}. The convolution operation between the pattern-aligned (strictly periodic target signal) time-frequency spectrogram, S[\hat{ω},\hat{t}], and the kernel K with H total harmonics at any specific frequency location, \hat{ω}, and time, \hat{t}, is formulated in Equation 18.

$$( S * K ) [ \hat{ω} , \hat{t} ] = {\sum }_{k = 1}^{H} {\sum }_{t = - T}^{T} S [ \hat{ω} + k {D}_{f} , \hat{t} - {D}_{t} t ] K [ k , t ] \qquad ( 18 )$$

In this embodiment, the method of the present disclosure has adapted the U-Net architecture as the foundation of the disclosed neural structure, while substituting standard convolution kernels with dilated harmonic convolutions. Moreover, two design principles are maintained for harmonic integrity. Firstly, pooling in the frequency dimension is prohibited, ensuring the size of this dimension remains unchanged throughout the network. Secondly, the dilation in frequency is set to 1 Hz stride (only towards higher frequencies) to promote the strict distance of the consecutive target's harmonics after pattern alignment, while suppressing noise and interference.

Previously, in FIG. 9A and FIG. 9B, the disclosed SpAc LU-NET structure and the function of the dilated harmonic convolution in time and frequency dimensions were shown. Given the application-specific setting of the spectrogram's resolution in both time and frequency, the parameters Dt and Df must be adjusted accordingly. The parameter Dt determines the temporal reach, which should be calibrated based on the prior knowledge on the dynamics of the target signal. A broader temporal reach is indicative of a more relaxed prior setup, enhancing smoothness, whereas a narrower reach is advantageous for capturing signals with rapid amplitude changes. In the context of the SpAc LU-Net architecture, the inclusion of each max-pooling layer doubles the temporal reach. Consequently, the adjustment of Dt must take into consideration the effect of two max-pooling layers within the described SpAc LU-Net framework.

### 5.4.3. Signal Separation by in-Painting

Masking and in-painting are utilized to separate sources in quasi-periodic mixed signals. At each round, a binary mask is utilized to conceal all significant harmonics of non-targeting sources from the cost function. The in-painting cost function is utilized for image in-painting applications. The selective visibility of the mask allows the optimizer to focus solely on the target source, in-painting the missing cross-overs and overlaps to retrieve the desired source. Equation 19 presents the in-painting cost function, wherein S_{mixed} and Sₒᵤₜ represent the mixed spectrogram and the neural network's output spectrogram, respectively.

$$E ( {S}_{out} ; {S}_{mixed} ) = {‖ mask * ( {S}_{out} - {S}_{mixed} ) ‖}^{2} \qquad ( 19 )$$

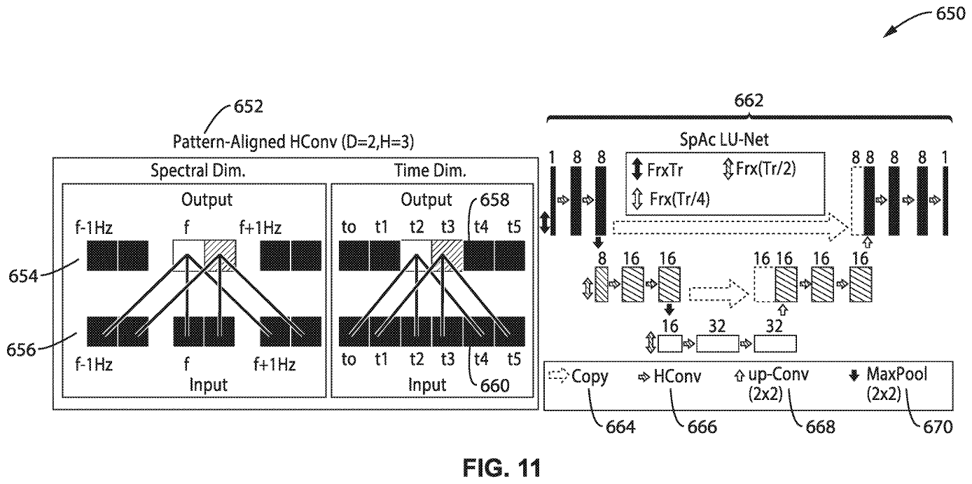

FIG. 11 illustrates example results 650 when investigating the effectiveness of the described neural network structure, SpAc LU-NET with harmonic convolutions, in its role as implicit priors for learning quasi-periodic time-frequency patterns. The figure shows patterned aligned HConversion 652 (D=2, H=3) with spectral domain outputs 654 mapped to 656, and time domain outputs 658 mapped to inputs 660. The process of SpAc LU-Net 662 is then shown from upper left processing FrxTr signals and performing HConvolutions 666, copy operations 664 along with MaxPool operation 670 to the center row of the figure as Frx(Tr/2), and to the bottom row of that figure as Frx(Tr/4), each of which also depicts H conversions and copies then up-conversions 668.

The method provides a comparative analysis of learning outcomes from different convolution kernels and network configurations receiving the same masked image. A comparison between conventional convolution kernels and harmonic convolution layers reveal the repetitive vertical frequency patterns, which are noticeable even at the initial stages of learning. However, the conventional baseline harmonic convolution approach which allowed for anchors larger than one, leading to inaccurate backward harmonic associations, and employed max-pooling in the frequency domain resulting in imprecise spectral representation, compromises the strength of the prior structure and elevates noise levels. In contrast, the disclosed adaptive harmonic priors surpasses traditional methods by promoting harmonics of the target at a fixed distance, aligned with the target frequency pattern after pattern alignment, and thus minimizing the effect of interference. Additionally, facilitating access to consecutive harmonics, which typically contain more energy than frequency bins at integral multiples of each bin, offers substantial benefits for in-painting of higher frequency components. Consequently, the present disclosure provides for a Spectrally Accurate design that exhibits significant noise reduction compared to other state of the art approaches. This improvement is particularly pronounced when employing a larger time dilation that further encourages the vertical target patterns over time.

### 5.4.3.1. Stable Training Under Interference

Multiple parameters can affect the performance of DHF signal separation. Two key factors are the masked duration of the target relative to the rate of amplitude change over time, and the energy ratio of the target relative to noise or interfering signals. Varying the overlap duration alters the minimum required time dilation for accessing a clean target signal that is visible during learning and can guide the optimization to convergence. More specifically, when a convolution kernel is applied to the center of the longest overlap, time dilation can be adjusted to access clean data before and after an overlap. However, since the network structure acts as an implicit prior, time dilation should be limited by a maximum threshold set based on the rate of signal variation over time. In the presence of a long overlap duration, where the minimum required dilation to access unmasked data exceeds the maximum threshold, a lower dilation setup can impair optimization stability and convergence. Another factor affecting DHF performance is the energy ratio of the target to noise or interference, which can also undermine stability and convergence if this ratio is small. In such cases, incorporating a secondary term in the cost function to enforce an explicit prior can aid convergence.

To further enhance optimization convergence, expanding the cost function to include some measure of the variation of energy per harmonic of the target over time is beneficial. Owing to the strict periodic harmonic pattern of the target after pattern alignment, penalizing the cost function with squared energy variation at select frequency bins, representing specific signal harmonics, is advantageous. This term contributes to enforcing smooth energy variation in masked areas with long duration; furthermore, it suppresses sudden energy jumps in the unmasked spectrogram regions due to noise and interference. Energy variation of the kth harmonic at time stamp \hat{t} and time increment Δt, assuming 2wₕ+1 bins carry the overall energy of the select harmonic at the specified time stamp, is expressed in Equation 20. Equation 21 presents the squared variation term, where Tₛ and nₕ represent the spectrogram time duration and number of considered harmonics.

$$V ( \hat{t} , k ) = {Δ}_{\hat{t}} [ {\sum }_{w = - {w}_{h}}^{{w}_{h}} {S}_{out} ( {kD}_{f} + w , \hat{t} ) ] \qquad ( 20 )$$

$$SqV = {\sum }_{\hat{t} = 1}^{{T}_{S}} {{\sum }_{k = 1}^{{n}_{h}} [ V ( \hat{t} , k ) ]}^{2} \qquad ( 21 )$$

However, introducing a squared variation term to the cost function too early can misdirect learning and trap optimization in a local minimum, where a relatively low energy, close to background levels, is assigned to the frequency bins of the target signal. To mitigate this issue, a dynamic weight is applied to the squared variation term that increases over optimization iterations. Equation 22 expresses the dynamic weight formulation, where aₜᵥ and bₜᵥ control the slope and position of the weight increase in relation to the optimization iteration number, nᵢ. By this definition, the cost function is presented in Equation 23.

$${L}_{tv} ( {n}_{i} ) = \frac{{w}_{m ax}}{1 + {e}^{- {a}_{tv} ( {n}_{i} - {b}_{tv} )}} \qquad ( 22 )$$

$$Cost = {w}_{0} E ( {S}_{out} ; {S}_{mixed} ) + {L}_{tv} ( {n}_{i} ) SqV ( {S}_{out} ) \qquad ( 23 )$$

### 5.4.4. Cyclic Phase Interpolation

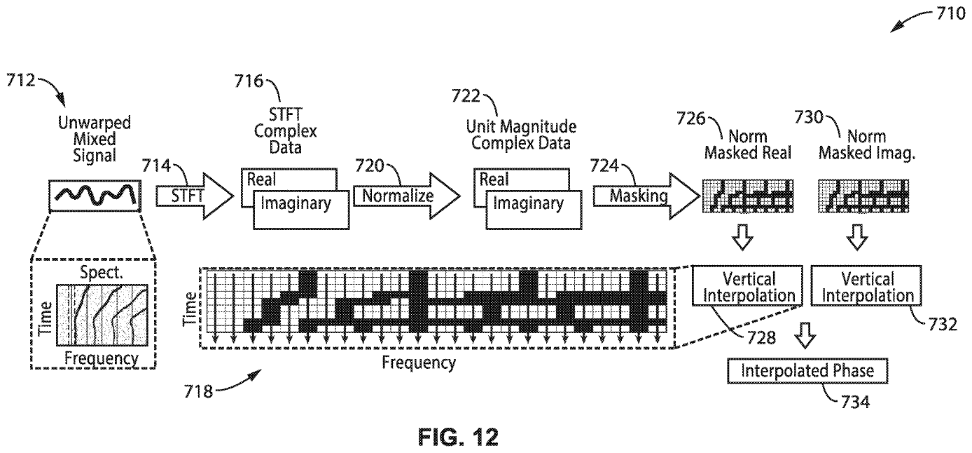

FIG. 12 illustrates an example embodiment 710 of the disclosed phase interpolation procedure. Phase interpolation of the STFT data is conducted independently from the spectrogram in-painting. The phase interpolation input signal is the pattern-aligned mixed signal 712, unwarped around the fundamental frequency of a specific target, and the generated mask that marks interpolation areas due to overlaps and cross-overs from other interfering signals or noise. Applying STFT transform 714, the initial complex data 716 contains both amplitude and phase information. To omit the amplitude information, amplitude normalization 720 is applied on the STFT data, setting an amplitude of one 722 for every complex data point. Utilizing the previously generated mask 724, to arrive at masking for the real 726 and imaginary 730 planes.

The phase is then estimated 728, 732 during overlaps and crossovers through interpolation. Owing to the strictly periodic patterns in pattern-aligned time-frequency space 718, each frequency bin is interpolated over time separately, and concurrently with other frequency bins. For each bin, both the real and imaginary components of the normalized masked data are interpolated, subsequently calculating the interpolated phase 734 by integrating these results; toward ensuring that the cyclic nature of the phase is preserved during interpolation.

### 5.5. Empirical Evaluation

In this section, performance of the extended DHF method is evaluated and compared to that of existing methods when applied to both synthesized and in-vivo TFO datasets. Initially, the specifics of the synthesized dataset are discussed, including empirical findings on the synthesized performance. Following this, the discussion explores how the DHF method performs using in-vivo data, which was gathered through experiments involving pregnant ewe models.

### 5.5.1. Synthesized Data

A tool for generating and characterizing task-specific synthesized quasi-periodic timeseries, g[n], with a sampling frequency of Fₛ and a total of np complete periods has been developed according to an embodiment herein. Each period is characterized by the desired input function per period, I(t), time duration per period list, D, and amplitude per period list, A, as given in Equations 24 and 25. The signal's shape and values for these settings can be input various formats, for example in either Comma-Separated-Values (CSVs) format or using an input image. W(t) in Equation 26 represents a windowing function to ensure that the first and last value of h are equal for continuity in the generated function.

$$D = [ {d}_{1} , {d}_{2} , {d}_{3} , … , {d}_{np} ] \qquad ( 24 )$$

$$A = [ {a}_{1} , {a}_{2} , {a}_{3} , … , {a}_{np} ] \qquad ( 25 )$$

$$h ( t ) = I ( t ) W ( t ) , t ∈ [ 0 , 2 π ) \qquad ( 26 )$$

Employing the phase, φ[n], defined earlier in Equation 15, the phase at each sampling time is computed by feeding the fₜₛ values derived from the inputted time duration list and sampling frequency (refer to Equation 27). Finally, g[n] is calculated using the windowed function and the phase information.

$${f}_{ts} = repeat ( \frac{1}{D} , D ⊙ {F}_{s} ) \qquad ( 27 )$$

$$a\_ts = repeat ( A , D ⊙ F\_s ) \qquad ( 28 )$$

$$g [ n ] = {a}_{ts} [ n ] h ( ( ϕ [ n ] + {ϕ}_{0} ) \% 2 π ) \qquad ( 29 )$$

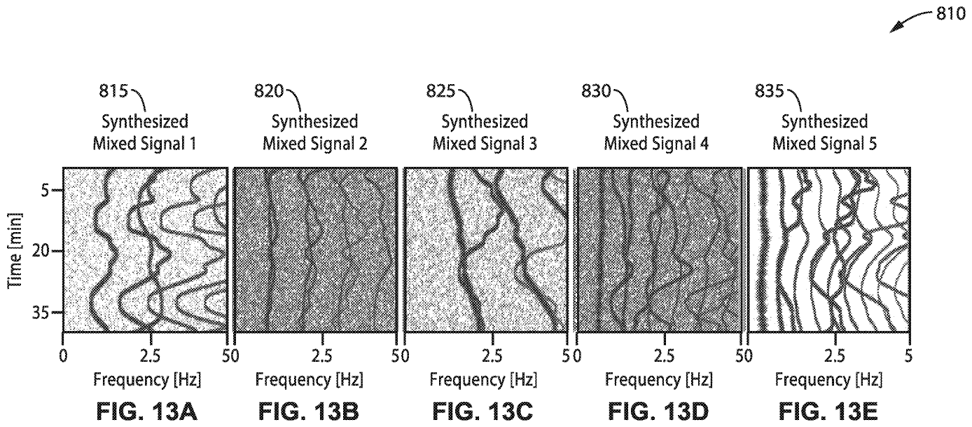

FIG. 13A through FIG. 13E illustrate spectrograms 810 of five distinct synthesized mixed quasi-periodic signals with sampling frequency of 100 for TFO application. Each mixed signal has 2-3 sources for respiration, maternal pulsation, and fetal pulsation, and an additional Gaussian noise. The respiration PPG shape is extracted from real sheep experiments after filtering out other dynamics. The pulsation PPG shape is randomly extracted from an MIMIC-IV dataset. The details of the synthesized signals are further explained in Table 4. Mixed Signals 1-3 each have two sources, maternal pulsation and fetal pulsation. The sources in Mixed Signal 1 815 show interference in the second harmonic of the target source, while Mixed Signal 2 820 has interference on the first harmonic. In Mixed Signal 3 825 the second source has less than ×0.1 of the dominant source's amplitude. Mixed Signals 4-5 830, 835 have three signals each, respiration, maternal pulsation, and fetal pulsation, with differences in overlap duration and complexity, and less than ×0.1 of amplitude on their third source compared to their first source.

### 5.5.2. Results on Synthesized Data

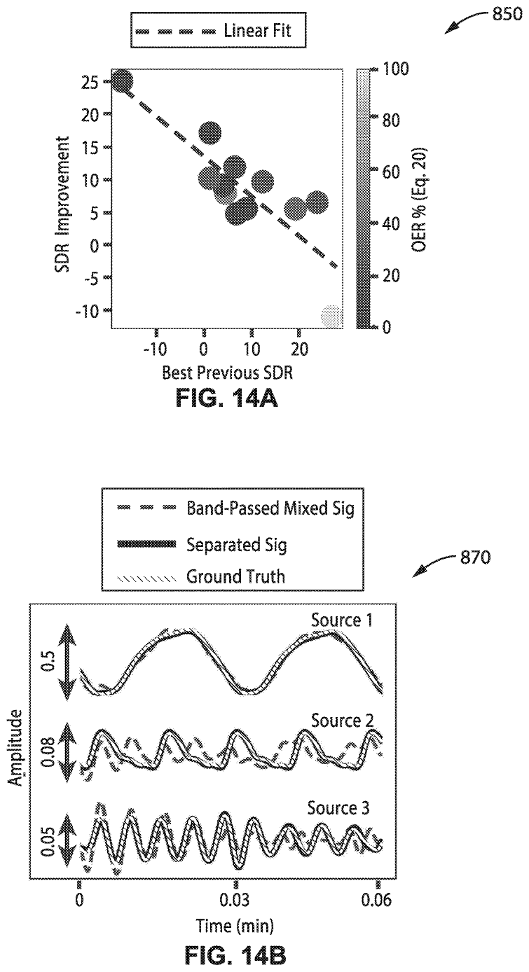

FIG. 14A and FIG. 14B illustrate an example of signal separation results when DHF is applied to mixed Signal 5 835 from FIG. 13E. In FIG. 14A is shown SDR improvement 850 in relation to best previous SDR. From this plot demonstrates notable improvement, particularly in more difficult cases. The waveforms 870 in FIG. 14B show a very close decomposition for all three sources (Source1 through Source3). The statistics of DHF performance are explored further in comparison to previous signal separation methods when applied on the generated mixed signals.

### 5.5.2.1. Metrics

The following presents Signal to Distortion Ratio (SDR) and Mean Squared Error (MSE) for each separated source from the five input mixed signals. For averaging MSE values, geometric averaging is employed, whereas for SDR averaging, arithmetic averaging is utilized in their original linear scale.

To further analyze the efficacy of DHF in various scenarios, two metrics are introduced: the Overlap Energy Ratio, OER, and Target Mask Duration Ratio, TMDR, as detailed in Equations 30 and 31. OER calculates the ratio of the masked target energy to total mixed-signal energy that overlaps with the target frequency, and TMDR is the ratio of target mask duration to overall signal duration providing a measure of target signal's visibility for in-painting.

$$OER \% = \frac{Masked Target Energy}{Overlap Mixed Energy} \times 100 \qquad ( 30 )$$

$$TMDR = \frac{Target Mask Duration}{Signal Duration} \qquad ( 31 )$$

### 5.5.2.2. Experiment Setup

The spectrograms are generated with window and stride of 60 s and 15 s. Data obtained from a comparison study showed that an increased time dilation parameter can improve the performance for extracting sources with longer masked sections. Commonly, the masked sections are longer when targeting sources with higher fundamental frequency than other sources in the mix. This is expected since the stride between harmonics of low-frequency sources are smaller and, therefore, the masked area on the spectrogram is larger. By way of example and not limitation, a dilation was employed of either 13 or 15, making the decision on a case-by-case basis according to specific masking situation.

Certain conventional signal separation algorithms, such as Independent Component Analysis (ICA) and Principal Component Analysis (PCA), do not accommodate single-detector signal separation. As a result, these conventional signal separation algorithms are excluded from the comparison study. The study compares against six previous signal separation methods, EMD, VMD, NMF, REPET and REPET-Extended (all described in previous sections), and Spectral Masking.

Spectral Masking is citation [5]: Timo Gerkmann and Emmanuel Vincent. 2018. Spectral masking and filtering. Audio source separation and speech enhancement (2018), 65-85.

The comparison results are presented in Table 5. These results are all calculated on band-pass filtered mixed signals (MSig) between [0 Hz, 12 Hz].

### 5.5.2.3. Discussion

The results in Table 5 demonstrate that the DHF method achieves an approximate 106% (3.15 db) improvement in Signal-to-Distortion Ratio (SDR) and 85% reduction in Mean Squared Error (MSE) on average, compared to the best previous signal separation algorithms. These results are particularly noteworthy for sources with less than ×0.1 amplitude of the dominant source, such as mixed signal 3 source 2, mixed signal 4 source 3, and mixed signal 5 source 3, where DHF demonstrated significant effectiveness. For these three low-power scenarios, the disclosed DHF method shows an average SDR improvement of 5.76 db and a 93% average MSE improvement over the best prior methods.

Referring again to the results of FIG. 14A and FIG. 14B analyzing SDR improvements achieved with DHF compared to the highest SDRs of previous methods. The findings indicate that DHF fills a critical gap in existing methods, particularly excelling in scenarios where others falter. FIG. 14A shows a heatmap 850 to illustrate the relationship between the Masked Energy Ratio (OER) and DHF's superior performance. Previous methods tend to struggle with low OER, attempting to isolate low-energy signals from strong overlapping interference, a challenge where DHF notably outperforms its predecessors. FIG. 14B shows an example 870 of a separated waveform after three rounds of DHF has been applied on a mixed signal with three sources.

Assuming knowledge of the fundamental frequency of each source gives our Deep Harmonic Filtering (DHF) technique an edge over previous approaches that do not incorporate this assumption. To evaluate the effectiveness of various signal separation methods, including this critical piece of information, a mask was first applied to retain only the target frequency signal, albeit with remaining overlaps and crossovers. Subsequently, we applied each signal separation method is applied to this pre-processed data. The results, as shown in Table 6, indicate that while some prior methods show some improvements after preprocessing, DHF consistently outperforms the competition keeping the aforementioned margin.

### 5.5.2.4. Performance in Constrained Timeframes

The nature of real-world health monitoring applications, where data is continuously streamed, necessitates the adaptation of signal separation techniques to work effectively within constrained timeframes. This subsection explores the performance of DHF when applied to shorter segments of data, specifically within the 2 to 5-minute windows. Such an investigation not only mirrors the practical challenges faced in real-world signal processing scenarios, but also highlights the robustness and adaptability of our methodology to operate under the pressing conditions of limited data availability.

A sliding window technique was employed on each synthesized mixed dataset to evaluate the performance of DHF under constrained time conditions. We experimented with two variations of the sliding window setup: firstly, a 5-minute frame duration with 2.5-minute sliding steps, generating spectrograms with a 30-second window size and a 5-second stride; and secondly, a 2.5-minute frame duration and 2.5-minute sliding steps, where spectrograms were generated using a 20-second window size and a 3-second stride.

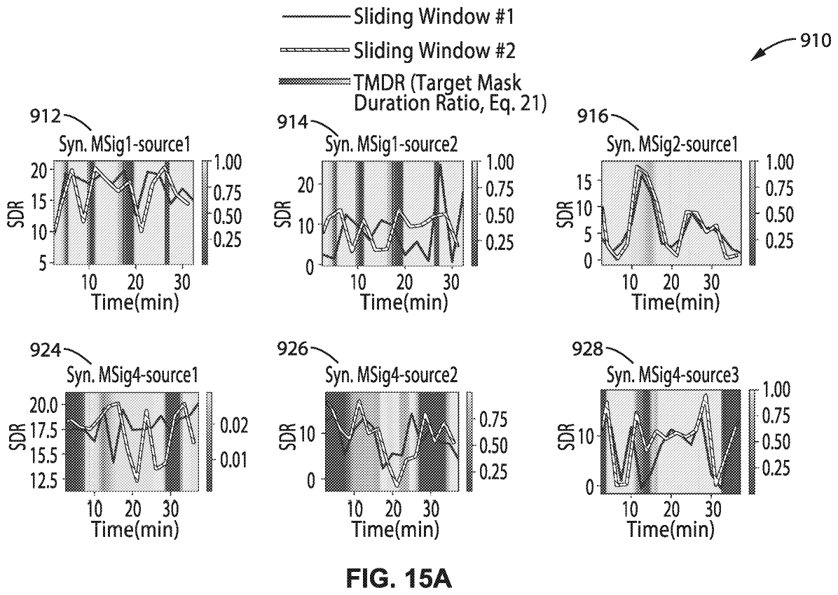

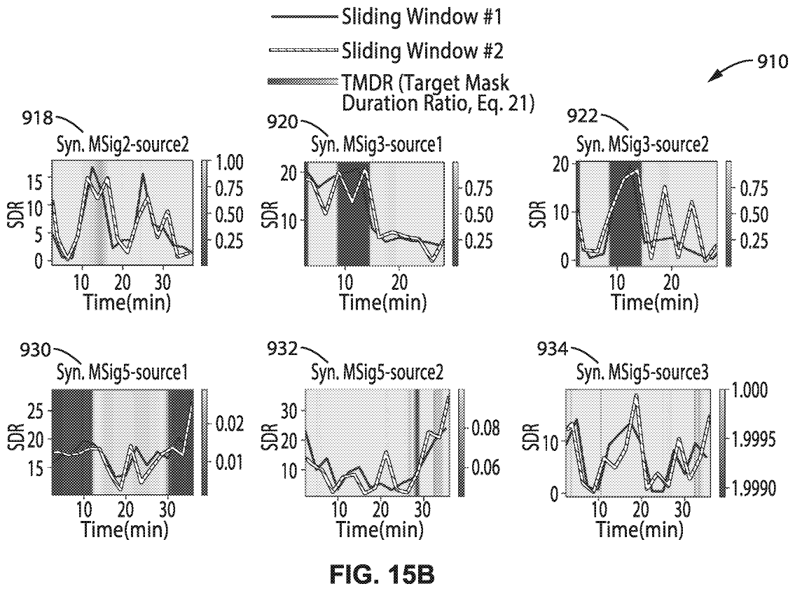

FIG. 15A and FIG. 15B illustrate the SDR achieved by DHF for each source, employing both sliding window configurations. These are rendered result images of this DHF performance study (SDR on y axis) when applied on five synthesized mixed signals with a constrained dataframe setup of 5-minutes (sliding window 1) and 2.5-minutes (sliding window 2). The background heatmap highlights TMDR parameter, which is a measure of target visibility in 1-minute sliding windows, where 0 and 1 show fully visible and fully masked respectively. The figures include a heatmap indicating TMDR parameter for each 1-minute frame duration, which effectively quantifies the extent to which the target is obscured by interference, ranging from 1 (completely masked) to 0 (fully visible).

The figure depicts 910 the results as follows: MSig1-source1 912, MSig1-source2 914, MSig2-source1 916, MSig2-source2 918, MSig3-source1 920, MSig3-source2 922, MSig4-source1 924, MSig4-source2 926, MSig4-source3 928, MSig5-source1 930, MSig5-source2 932, and MSig5-source3 934.

The results underscore that SDR improves as the visibility of the target energy increases. Conversely, when the target becomes less visible, the behavior of SDR is commonly reduced with higher variation over time. A comparison between the two sliding window setups indicates that a smaller frame size leads to a more pronounced increase in SDR when the target energy is visible, yet it also results in a quicker decline in SDR as soon as this visibility diminishes. This nuanced observation underscores the delicate balance between frame size and the ability to maintain high separation performance in conditions where target visibility is challenged. In such scenarios, a dynamic frame duration that can extend the use of previous data can help with SDR improvement.

### 5.5.2.5. Impact of Interference Energy and Duration on DHF Performance

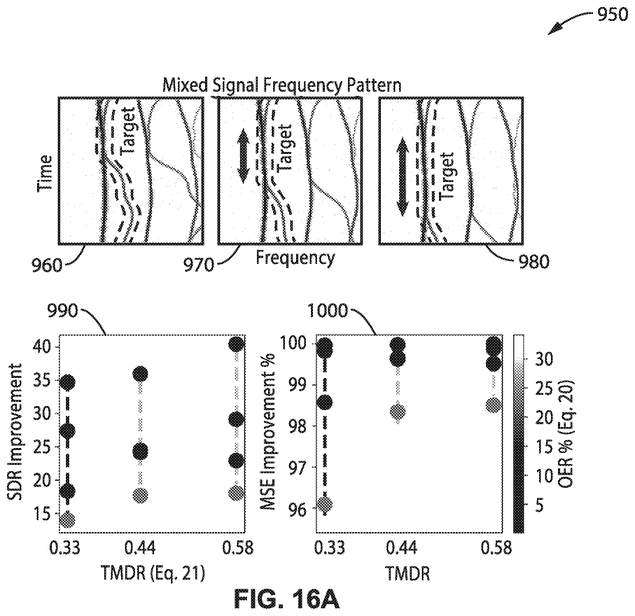

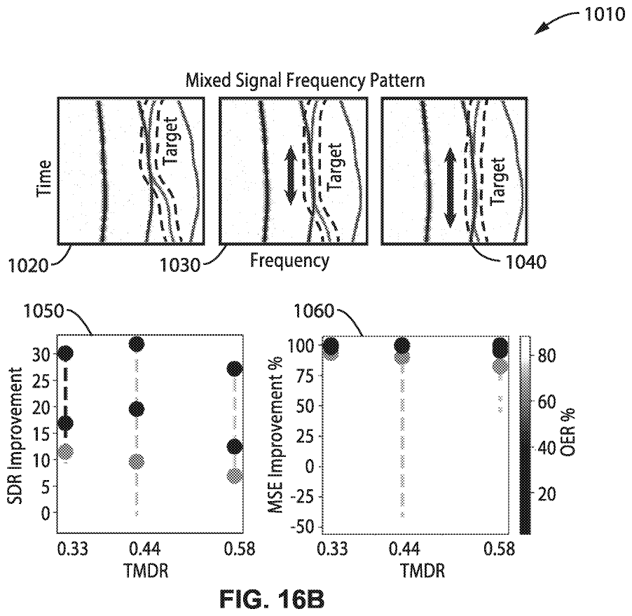

FIG. 16A and FIG. 16B illustrate plots 950, 1010 of performance results as DHF varies with changes in two key variables, namely the overlap duration and the energy ratio of the target signal relative to interfering signals. The DHF results are compared to best previous methods while varying overlap duration, shown with three lines, and overall energy of the target signal over interference energy, horizontal axis, when in FIG. 16A the overlap is on the first harmonic; and in FIG. 16B when the overlap is on the second harmonic. The fundamental frequency of the target signal is highlighted with dash lines in the spectrographic image.

By maintaining similar amplitude levels across different training scenarios, the parameters are adjusted for overlap duration and total energy ratio, with three and five distinct settings for overlap duration and energy ratio, respectively. In FIG. 16A the overlap affects the first harmonic of both the target and interfering signals; whereas in FIG. 16B, the overlap is on the second harmonic of the interfering signal. It was observed that the performance of previous methods improves when the overlaps do not occur at the mutual fundamental frequency, and thus relatively less improvement is seen in these instances when compared to the harder cases where fundamental frequencies overlap. Focusing on the more challenging setup of FIG. 16A, DHF shows a notable improvement in performance as the overlap duration increases, and thus TMDR increases, outperforming previous techniques that struggle to separate the sources accurately. DHF maintains steady performance gains at lower overlap energy ratios (OER). As OER increases, however, making signal separation less challenging for existing methods, the performance gains observed with DHF become more variable. For instance, FIG. 16B demonstrates this at TMDR of 0.44 and OER larger than 85%.

### 5.5.3. Results on In-Vivo Measured Data

Fetal SpO₂ estimation accuracy using a transabdominal photoplysmograph (PPG) dataset acquired from two term-pregnant ewes is now discussed. The dataset consisted of 40 minutes of continuous mixed PPG signals at 740 nm and 850 nm wavelengths gathered from a pregnant ewe's abdomen and ground-truth fetal blood oxygen saturation readings, i.e., SaO₂, measured from blood-draws with the time distance of 2.5, 5, and 10 minutes.

Referring back to FIG. 6A a TFO data acquisition method and results were previously shown for the adopted dataset. Since the ground-truth fetal PPG signal is not accessible, the correlation of SpO₂ estimation was reported with measured SaO₂ readings when the fetal signal is separated using spectral masking and DHF method. This is compared against spectral masking because it has shown the best performance among previous works.

### 5.5.3.1. Fetal Oxygen Saturation Estimation Method

After separating the fetal signal from PPG readings, collected in sheep's TFO study, through the described DHF, a non-invasive estimation was made of fetal blood oxygen saturation through pulse oximetry (SpO₂). The separated fetal information at a high level is required for calculation of the fetal modulation ratio, R, as described in Equation 32. Fetal modulation ratio is a measure of light absorption changes in the abdominal near infrared spectroscopy, for two measurement wavelengths (λ₁ and λ₂), due to fetal pulsatile and arterial blood flow. AC and DC parameters in this concept present the dynamic and static parts of fetal PPG signal. Equation 33 shows how modulation ratio can be used to estimate SpO₂. In this equation K is a constant and v₀ and v₁ are optimization parameters.

$$R = \frac{{( AC / DC )}^{{λ}_{1}}}{{( AC / DC )}^{{λ}_{2}}} \qquad ( 32 )$$

$${Sp O}_{2} = - k + \frac{1}{{v}_{o} + {v}_{1} R} \qquad ( 33 )$$

Equation 34 presents the employed linear regression method for SpO₂ estimation given Equation 33 and the ground truth fetal arterial oxygen saturation, SaO₂, through regular blood draws. Value k is a constant regularizing term and v₀ and v₁ are the learned parameters. In this work, similar R values are averaged over a 2.5 minute window centered around each blood draw time stamp.

$${Y}^{′} = \frac{1}{Sa O 2 + k} = {v}_{0} + {v}_{1} R , k = 1.885 \qquad ( 34 )$$

### 5.5.3.2. Discussion

Referring back to FIG. 6B and FIG. 6C SpO₂ estimation performance was previously shown for both sheep, comparing the results when the separated fetal signal is obtained through spectral masking (similar to) and DHF. Comparing the correlation between SpO₂ estimations and SaO₂ readings, the extended DHF method improves the correlation from 0.24 to 0.81 and from 0.44 to 0.92 in sheep 1 and sheep 2, respectively (80.5% average error improvement from ideal correlation of 1 between SpO₂ estimations and SaO₂ readings).

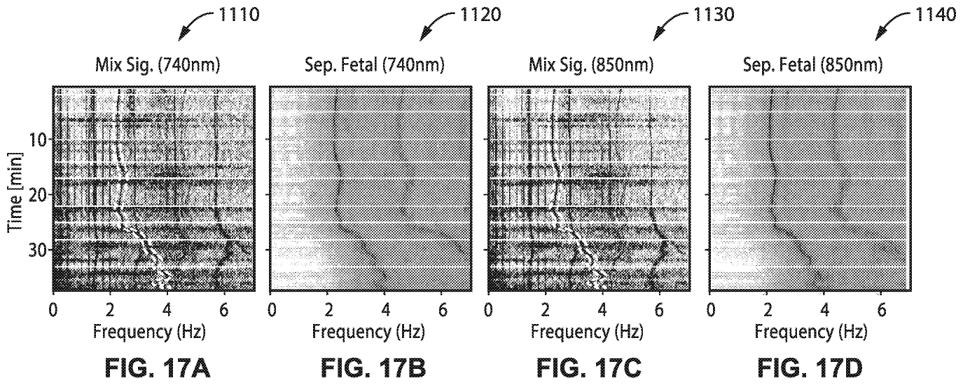

FIG. 17A through FIG. 17D are examples of measured mixed signal spectrograms for sheep 2 at 740 nm 1110 and 850 nm 1120 and the separated fetal signal 1130, 1140 at each wavelength using the DHF method. The horizontal lines mark blood draw timestamps, and dashed vertical lines highlight the fundamental component of fetal lamb's heartbeat signal before separation.

### 5.6. Conclusion on DFE Extended

Limitations in dataset quality and quantity, present in many quasi-periodic signal separation applications in wearable systems, have hindered their success despite their significant potential. Incorporation of application-specific prior knowledge can enhance signal separation performance with limited data. In some embodiments, source frequencies were used, captured through additional sensors or posterior analytical methods; to first mask the undesired frequencies for a specific target source, and second to transform the time-frequency representation to create a stronger alignment between the target source and implicit deep priors built into the structure of the neural network. The method was showcased in the TFO application, using both synthesized and in-vivo data, where it shows significant improvement compared to the state of the art.

### 6. HarPE: A Generative AI Algorithm for Sample Efficient Signal Separation in Wearable Biosensors

### 6.1. Introduction to Harmonic Priors Ensemble (HarPE)

A number of wearable biosensors, such as those used in blood glucose monitoring or deep tissue sensing, face the challenge of extracting a signal of interest from limited noisy observations, which are confounded by concurrent physiological phenomenon. Biosensor data contains unique features conducive to signal separation. Notably, the frequency of interfering quasi-periodic signals, but not their amplitude, is often accessible either through processing of the sensed data or auxiliary modalities.

In this section of the disclosure is introduced a method referred to as Harmonic Priors Ensemble (HarPE), that separates quasi-periodic signals iteratively by masking the interfering signals in time-frequency space and restoring the target through deep prior in-painting. Drawing inspiration from the Deep Harmonic Finesse technique (DHF) described in previous sections of this disclosure, HarPE only requires a single data sample, and no additional training data, while advancing the state of the art in three aspects. (1) Utilizing a deep ensemble learning method to drastically improve amplitude estimation accuracy and manage noise effectively. (2) Addressing the challenge posed by sparse pixels in the target signal in time-frequency space, to enhance learning stability and accuracy, as well as reducing computation load and learning cost. (3) Embedding priors in the cost function to preserve the persistent pulsating pattern of the target physiological signal and enhance convergence during prolonged periods of interference and noise. Results on this method using both synthesized data and in-vivo animal studies demonstrate significant performance benefits to the use of HarPE over current state of the art methodologies.

### 6.2. Related Work

Single-channel signal separation is a crucial task in various fields, traditionally addressed through analytical methods, and more recently through machine learning techniques. The simplified modeling in traditional methods lacks the flexibility to accurately model complex real-world data. Advanced deep learning approaches offer greater flexibility and variability in their models, yet often require substantial amounts of training data, that is impractical in many applications.

Traditional single-channel signal separation methods like Empirical Mode Decomposition (EMD) and Variational Mode Decomposition (VMD) break down signals into Intrinsic Mode Functions (IMFs) based on local dynamics or by solving variational problems for enhanced frequency separation. Non-negative Matrix Factorization (NMF) further decomposes signals across time and frequency domains, while the REpeating Pattern Extraction Technique (REPET) and its advanced form, REPET-Extended, play key roles in audio by isolating sounds through recognition of repeating patterns.

Deep learning approaches have shown significant impact on handling large, labeled datasets in audio separation. However, their application in biosensors is limited by sparse and noisy available data. Deep prior techniques tackle the challenge of learning with limited data by embedding implicit priors within their neural structures, and learning with only a single data sample and no additional training data. One of these approaches employs a deep convolutional prior structure and separates sound by exploiting auditory coherence and discontinuity, but is not applicable on continuous quasi-periodic signals. Another approach is a new harmonic convolution structure as deep audio prior for audio processing. The techniques of ensemble learning reduce bias and variance by utilizing multiple models, and thus, enhance performance.

### 6.3. Illustrative Application

A notable approach is boosting methods sequentially to correct errors by focusing each learner on errors from previous stage, thus controlling variance and enhancing robustness. This portion of the present disclosure describes integrating gradient boosting with deep prior techniques, promising significant advancements in signal separation.

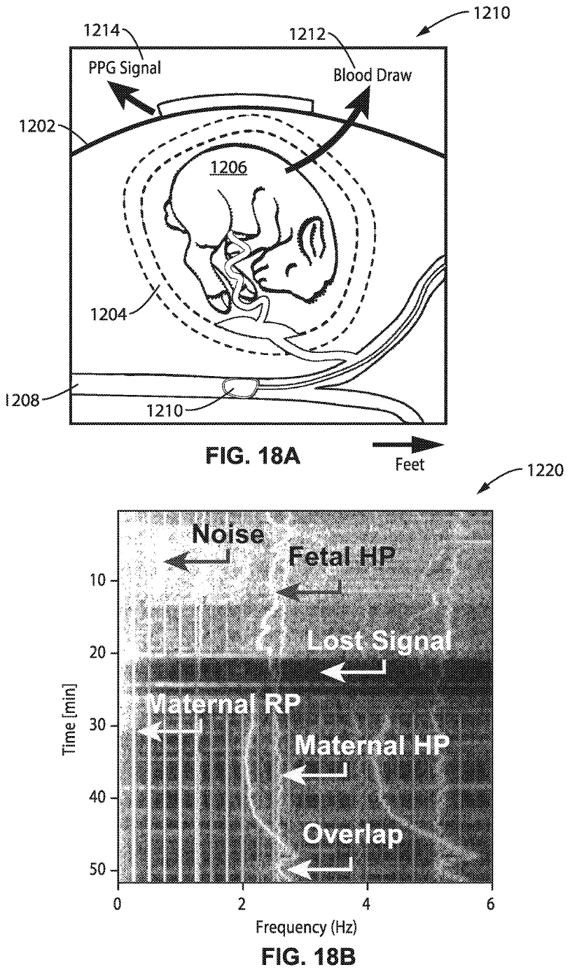

FIG. 18A and FIG. 18B illustrates TFO data collection 1210 in pregnant bovine models, and a time-frequency spectrogram 1220 of an example sensed mixed PPG signal. In the figure is seen the abdomen 1202 of the ewe with uterus 1204, and ewe fetus 1206, with a pulse oximeter 1210 inserted into the Aorta 1208 of the ewe. Blood draw 1212 and PPG signals 1214 are represented as well.

Transabdominal Fetal Oximetry (TFO) is a non-invasive method for estimating fetal arterial blood oxygen saturation, devised to complement traditional Cardiotocography (CTG) for intrapartum fetal monitoring. While CTG effectively identifies fetuses at risk of birth asphyxia, its high false positive rate can lead to unnecessary interventions, such as emergency Cesarean surgeries. By providing continuous monitoring of fetal blood oxygen saturation, TFO aims to enhance the accuracy of fetal assessments, thereby improving outcomes for both mothers and neonates.

TFO operates via non-invasive measurement of diffuse reflectance near infrared light. Unlike conventional pulse oximetry, the captured Photoplethysmography (PPG) signals contain a mixture of information from both superficial maternal and deep fetal tissue layers. Thus, separation of signal sources contributing to the sensed mixed signal is an essential precursor to analyzing changes in light absorption due to the variations in the fetal blood oxygen saturation (sensing target).

The example PPG spectrogram motivates the need for isolating quasi-periodic sources from a sensed mixed signal, in particular, when the target signal of interest has low SNR, or is overshadowed by considerably stronger structured contamination. It should be recognized that as light exponentially attenuates in tissue relative to the traveled distance, the sensed intensity changes due to fetal heart rate are significantly weaker, such as up to 5 orders of magnitude, than those due to the maternal tissue. In this context, the performance advantages of HarPE are demonstrated for use in fetal signal separation, while utilizing only a single data sample without the need for additional training data. It should, however, be appreciated that the HarPE method can be applied to a wide range of signal separation applications and is not limited to the described fetal monitoring application.

### 6.4. Methodology

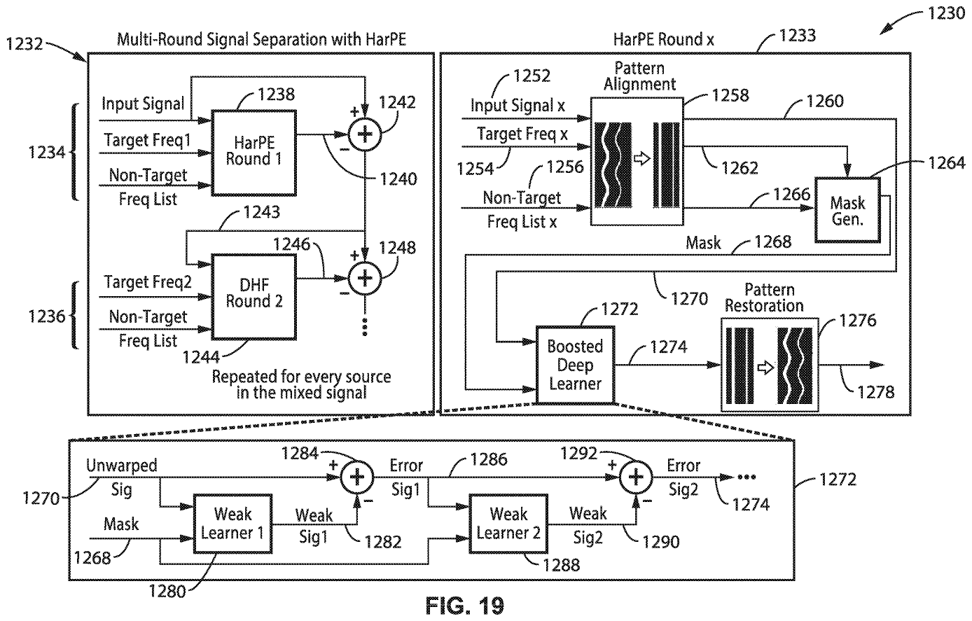

FIG. 19 illustrates an example embodiment 1230 of the disclosed method in which multiple rounds 1232 of signal separation are performed. Each HarPE round 1233 corresponds to separation of one target signal from the mix. HarPE takes an iterative approach to high-precision separation of quasi-periodic signals, with known potentially-overlapping frequencies, but unknown amplitudes and phases, from a given mixed signal. It processes the mixed signal in 2-D time-frequency space.

By way of example two rounds 1232 are shown 1234, 1236, each round receiving an input/residual from the previous round, a target frequency, and a non-target frequency list. As in DHF, the input signal is processed in Round1 1238 and this output 1240 is subtracted 1242 from the input signal to generate residual 1243 which is directed to the input of Round2 1244 and from which the output 1246 of Round2 is subtracted 1248. Then this process is repeated for each of the rounds.

Looking now at the stages in one HarPE round in which each iteration isolates one target signal by masking interfering sources, and restoring the target through generative in-painting. Received is an input signal x (for roundx) 1252, target frequency x 1254, and non-target frequency list x 1256, and outputting signals. These signals are received for aligning 1258 target harmonic patterns in time-frequency space to expected patterns in the deep neural prior model, and outputting an unwarped signal using target x 1260, which is directed to a boosted deep learner 1272. Pattern alignment signals of Strictly Periodic Target Frequency 1262, and an unwarped non-target frequency list 1266 are directed to a mask generator 1264 which outputs mask 1268 to the boosted deep learner 1272. The output 1274 of the boosted deep learner is processed for pattern restoration 1276 with output 1278. The operation of the boosted deep learner 1272 is shown at the bottom of the page with receiving unwarped signal 1270, and mask 1268 to Weak Learner1 1280, with the mask passing on through to Weak Learner2 1288. Weak Signal1 1282 is subtracted from the Unwarped Signal to generate Error Signal 1286 as another input to Weak Learner2 1288. Weak Signal2 is subtracted from Error Signal1 to result in Error Signal2 1274 and the process can continue.

The following describes these elements with more particularity. Thus, the iteration begins with aligning target harmonic patterns in time-frequency space to expected patterns in the deep neural prior model, followed by masking and in-painting interference in the transformed space using a chain of weak learners, implementing boosted in-painting. After each separation, the residual signal is passed to the next round of HarPE to isolate subsequent signals.

### 6.4.1. Pattern Alignment Preprocessing

To align the target patterns of the mixed-signal in time-frequency space with the deep prior model, a pattern alignment unit preprocesses the input and unwarps it relative to the frequency patterns of the target signal. This unit resamples and transforms the input from a steady-time sampling space to a target-specific steady-phase sampling space, where the target signal's frequency pattern becomes strictly periodic.

Equation 35 defines the input mixed signal, X, as sum of multiple quasi-periodic sources plus noise, and Equation 36 calculates the phase of the target signal, Φₜₛ, at each time step n. Here, aᵢ[n] and fᵢ[n] represent the variable amplitude and frequency of source i, with Δt as the sampling period.

$$X [ n ] = {\sum }_{i} {a}_{i} [ n ] cos ( 2 π {f}_{i} [ n ] n Δ t + {Φ}_{i , 0} ) + noise \qquad ( 35 )$$

$${Φ}_{ts} [ n ] = 2 π Δt {\sum }_{i = 1}^{n} {f}_{ts} [ i ] + {Φ}_{ts , 0} \qquad ( 36 )$$

Receiving the dynamic frequency pattern of the target source, fₜₛ[n], the pattern alignment unit resamples the mixed signal to enforce linear target phase progression in the transformed space. This translates to a constant target fundamental frequency based on Equation 36.

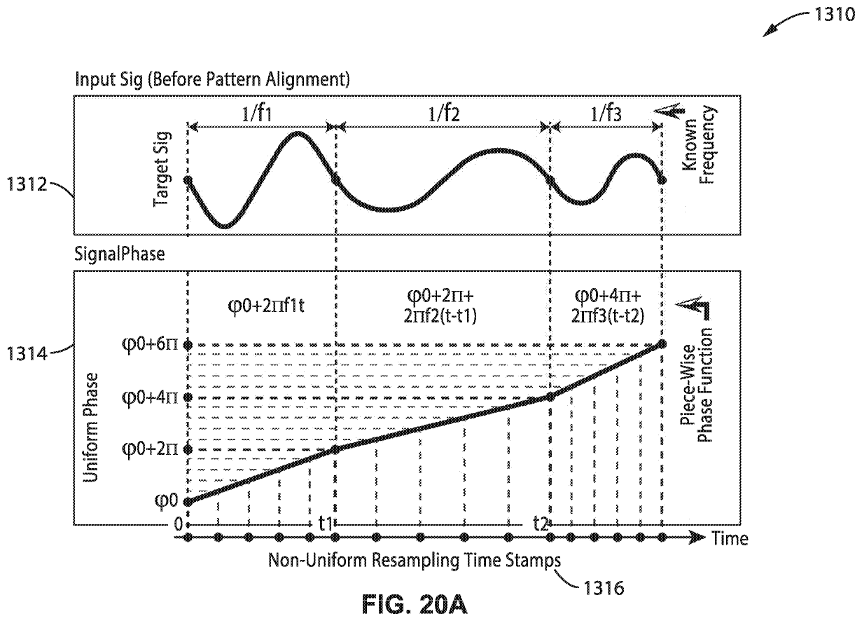

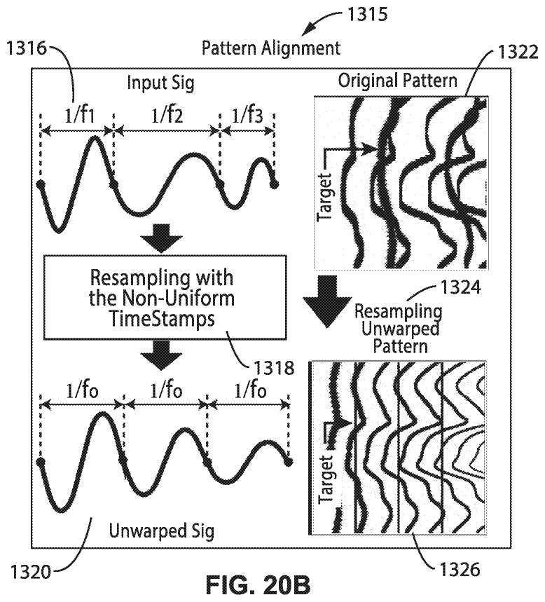

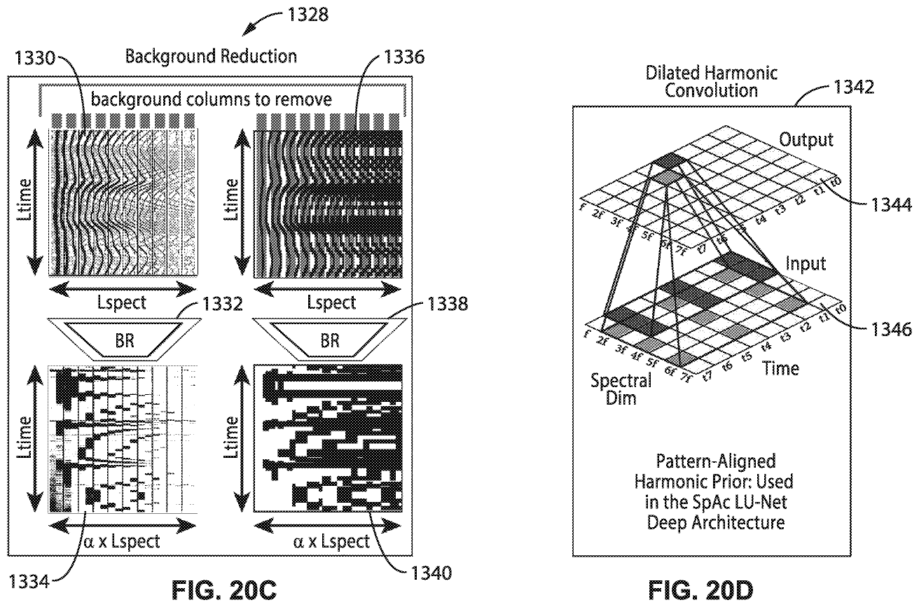

FIG. 20A through FIG. 20D illustrate the pattern alignment procedure and its effect on the time-frequency representation. FIG. 20A screen 1310 shows input signal 1312 before pattern alignment, and the Signal phase 1314 showing piece-wise phase functions and the non-uniform resampling time stamps 1316. FIG. 20B shows pattern alignment 1315, with input signal 1316, resampling with the non-uniform timestamps 1318 and the unwarped signal 1320. Also is shown the original pattern 1322 with target signal identified, with resampling 1324 and the unwarped pattern 1326 with the target signal also identified. FIG. 20C shows Background Reduction 1328 showing background columns to remove 1330, 1336, Background Reduction (BR) processes 1320, 1338, and results 1334, 1340. FIG. 20D shows Dilated Harmonic Convolution 1342 with a pattern-aligned harmonic prior which is used in the SpAc LU-Net Deep Architecture which is showing input 1346 and output 1344 with their spectral and time dimensions.

Accordingly, the resampling process involves identifying time stamps that correspond to linear phase increments at a consistent stride of 40, ensuring a constant number of samples per full target pulsation period regardless of frequency dynamics. After unwarping, each target harmonic is consistently placed in the same spectral bin position over time, simplifying the prior pattern. After signal isolation, the original time samples are reused in the pattern restoration block to convert the strictly periodic target patterns back into their original quasi-periodic space.

### 6.4.2. Deep Prior Model

In deep prior learning, the structure of the neural model acts as implicit prior to extract patterns of the input data over noise, without using any additional training data.

In FIG. 20A through FIG. 20D was shown utilizing SpAc LU-Net, in a multi-scale UNet architecture that employs dilated harmonic convolution kernels for the task of spectrogram in-painting in the time-frequency domain. These kernels adjust the receptive field in the spectral dimension to access integral multiples of each output frequency bin, corresponding to the harmonics of that specific bin. This approach not only enhances the direct relevance of energy levels between harmonics of a signal but also reduces noise, which typically does not follow the harmonic pattern. In the time dimension, the dilated harmonic convolution further benefits from the strictly periodic pattern of the target source, achieved by pattern alignment preprocessing, where neighborhood in the time dimension is implemented through dilation. Equation 37 explains the harmonic convolution output at spectral bin \hat{ω} and time \hat{t} when convolution kernel K with time dilation Dₜ and H harmonics is applied to the input.

$${Out}_{conv} [ \hat{ω} , \hat{t} ] = {\sum }_{k = 1}^{H} {\sum }_{t = - T}^{T} X [ k \hat{ω} , \hat{t} - {D}_{t} t ] K [ k , t ] \qquad ( 37 )$$

To maintain the accuracy of harmonic access in the spectral dimension, SpAc LU-Net preserves the spectral feature size throughout the network. Therefore, max-pooling and up-convolutions are applied exclusively to the time dimension.

### 6.4.3. Boosted Deep Learner

The use of time-dilation and MaxPooling layers in the SpAc LU-Net network architecture creates a dilated receptive field, which often leads to the over-smoothing of signal components and increased errors in signal separation. This effect is particularly detrimental when dealing with signals characterized by rapidly changing and irregular amplitudes, where capturing fine details are essential.

To counteract this and improve estimation accuracy, a deep boosting technique is introduced that leverages an ensemble of weak neural networks. This approach uses a cascading arrangement, where each network in the sequence is tasked with estimating the residual signal left from its predecessor. This method reduces the high bias of the designed neural network and helps with generalizable learning, while controlling the variance for accurate learning of nuances and fine details of the data. This equips the model to handle signals with complex, rapidly varying amplitude spectra more effectively.

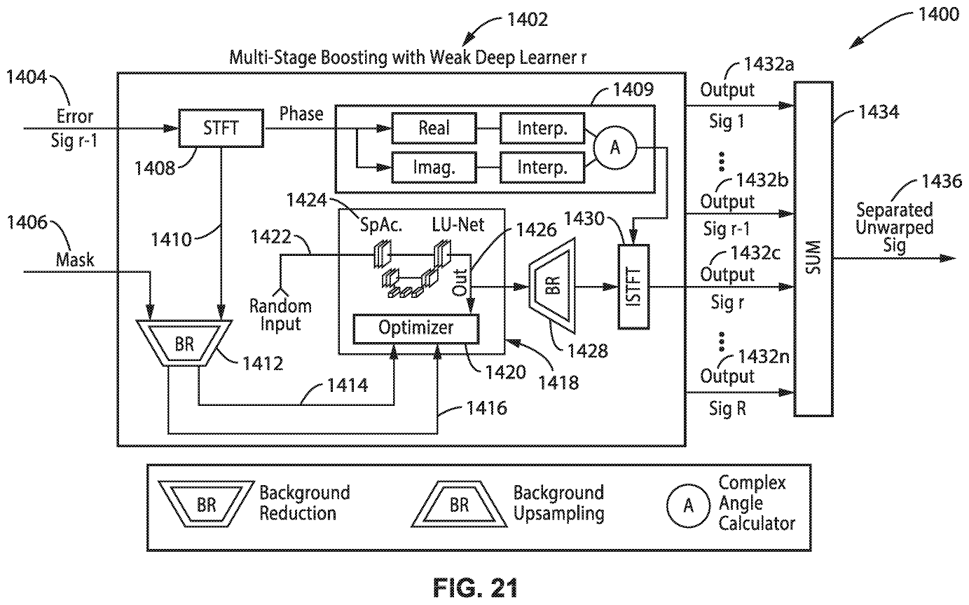

FIG. 21 illustrates the boosting process 1400 within the HarPE signal separation framework using Weak Deep Learner ‘r’ 1402. Each weak neural network in the cascade is initialized using the weights from the preceding network, while using a similar/reduced learning rate. Moreover, each network receives a rescaled error signal 1404 in the time-frequency space that set the range of energy variation. This rescaling is critical as it helps in accentuating the errors that need to be corrected in subsequent rounds, thereby enhancing the overall accuracy of the separation.

The figure shows Error Signal r-1 1404 to STFT 1408 which outputs phrase to Background Reduction (BR) 1412, and to a Cyclic Phase Interpolator 1409, with Real and Imaginary components and Interpolations arriving at a Complex Angle Calculator which output is directed to STFT 1430. Mask 1406 is also received at BR 1412 which outputs a Focused Spectrum signal 1414, and Focused Mask signal 1416 to an optimizer of the SpAc LU Net 1424, which also receives a Random Input 1422, and whose output is directed for BackGround Up Sampling 1428 before Inverse STFT 1430 and generating output Sig ‘r’ 1432c, which is shown with other outputs 1432a through to 1432n, these being all summed 1434 to provide the Separated Unwarped Signal 1436.

### 6.4.4. Balancing Foreground and Background

In HarPE, the harmonics of the target signal are the primary elements to restore, yet they represent only a small fraction of the total spectrogram of pixels. Achieving higher frequency resolution, necessary for accuracy, also increases the number of background pixels, which can dominate the cost function and impede the separation process. This is particularly problematic when dealing with low-energy target signals near the noise floor, as it can lead to an overemphasis on noise, compromising the accuracy and stability of signal reconstruction.

To overcome these challenges, the background's impact on the cost function is reduced through selective DownSampling of the time-frequency spectrogram, preserving only the essential margins around each target harmonic while discarding the excess background during background reduction stage. The spectral mask is similarly DownSampled to align with the spectrogram dimensions. In FIG. 20A through FIG. 20D was illustrated the background reduction procedure. After in-painting optimization with the reduced spectrogram, the removed background bins are restored in the UpSampling stage by filling them with the average background energy from the unmasked sections.

This approach reduces the input size and the computation load. Further, it enhances the model's ability to accurately restore signals with low energy levels close to noise. Overall, this strategy accelerates learning, improves stability, and significantly boosts the accuracy of target signal restoration.

### 6.4.5. Cost Function

The utilized cost function is composed of three weighted terms: in-painting, non-interrupted pulsation, and variation smoothing. Each term, discussed below, plays a critical role.

(1) In-Painting Term: In the deep prior in-painting method, a neural network receives a two-dimensional noise input and is tasked with generating an output that aligns closely with known pixels in a masked image. The cost function drives the network to minimize the difference between its output and the unmasked regions of the input, effectively leveraging the implicit priors encoded in the network's structure. Equation 38 shows the inpainting loss term, where the input mixed signal spectrogram and network's output spectrogram is presented as Iᵢₙₚ and Iₒᵤₜ, respectively. Masking is applied through pixel-wise multiplication of a two-dimensional binary mask with the spectrogram error. The targeted masking enables the deep network to restore the target signal by focusing on regions unaffected by interference, effectively mitigating overlaps and crossovers from other pulsating signals or noise sources.

$$L 1 = {‖ mask * ( {I}_{out} - {I}_{inp} ) ‖}^{2} \qquad ( 38 )$$

(2) Non-Interrupted Pulsation Term: In biosensor signals, it can be assumed that the ground truth target source is persistently pulsating and present, even when it is occasionally obscured by interference sources including other quasi-periodic signals, environmental noise, pathological variations, or measurement errors. However, the baseline cost function does not directly enforce signal persistence under mask, but rather relies on the network's structural prior to interpolate the missing information. Although this method typically maintains a target signal trace for shorter masked segments, it tends to gradually diminish the energy of the target signal during prolonged interference.

Considering the initial assumption of pulsation persistence, an additional cost function term is introduced in Equation 39 for improved signal restoration during masked intervals. This term enforces a threshold on the interpolated sections of the target source by penalizing the optimizer when the generated target signal diminishes. This threshold is calculated for each frequency bin of the target harmonics by averaging the visible amplitudes, permitting a predefined deviation percentage. Then, a saturation function σ, namely a sharp Sigmoid function centered around each spectral bin's threshold, saturates the estimated energies towards zero and one for values less or more than the threshold of the spectral bin, Rⱼ, respectively. A logarithmic loss function drastically penalizes the zero values, representing amplitudes less than the minimum threshold, to encourage values more than the minimum threshold over time in spectral bins that belong to target harmonics, bₜₐᵣ. While a step function is utilized to allow late introduction of the term to the cost function, one can use a more gradual approach if the application demands.

$$L 2 = - {\sum }_{j ∈ {b}_{tar}} {\sum }_{i} log ( σ ( {I}_{out , i , j} - {R}_{j} ) ) {W}_{G} \qquad ( 39 )$$

$${W}_{G} = step ( {n}_{epoch} - {n}_{x} ) \qquad ( 40 )$$

(3) Variation Smoothing Term: During spectrogram restoration, it is crucial to maintain the continuity and smooth variation of the fast Fourier transform energies from one time window to the next, which should follow the application-specific expected rate of change. To achieve this, total variation loss that minimizes abrupt changes and reduces estimation noise between consecutive spectral bin pixels is employed within a given time window. The pattern alignment unit plays a pivotal role in simplifying the application of total variation term, as it allows for consistent access to the same target signal pixels over time in the same spectral location.

Equation 41 defines the total variation term applied to the target spectral bins. The weighting of this term is dynamically adjusted based on the epoch number, n_{epoch}. This approach allows for a gradual introduction of the smoothing effect, controlled by the parameters as and bs.

$$L 3 = {W}_{dyn} {\sum }_{j ∈ {b}_{tar}} {\sum }_{i} {( {I}_{out} ( i + 1 , j ) - {I}_{out} ( i , j ) )}^{2} \qquad ( 41 )$$

$${W}_{dyn} = \frac{1}{1 + {e}^{- {a}_{s} ( {n}_{epoch} - {b}_{s} )}} \qquad ( 42 )$$

### 6.4.6. Cyclic Phase Interpolation

To restore the target signal after masking interference, both spectrogram energy and phase of the short time Fourier transfer must be interpolated. The spectrogram in-painting is performed using the described deep prior methodology, and the phase interpolation is performed separately using a cyclic interpolator. To preserve the cyclic range of phase, the phase is normalized and its real and imaginary parts are interpolated separately. Given the strictly periodic pattern of the target on the short time Fourier transform, the variation of each spectral bin over time is interpolated separately from other bins. The interpolated phase is used alongside the in-painted spectrogram for inverse transition from time-frequency space to a time-series signal.

### 6.5. Experimental Results

The following presents results using both synthesized data, and mixed signals collected within in-vivo animal experiments.

### 6.5.1. Experimental Setup

Spectrograms were generated using a 60-second window with a 10-second stride. Each round of separation utilized two consecutive weak learners, unless specified otherwise. The first weak learner is initialized with random parameters and trained with a learning rate of 1e-3 for 2500 epochs. The non-interrupted pulsation loss was applied only at this stage. Subsequent weak learners are initialized using the parameters learned from the preceding weak learner, with a reduced learning rate of 5e-4, and trained for 2500 epochs. The weights for the cost function components were optimized empirically. Constants aₛ and bₛ in Equation 42 were set to 1e-3 and 1000, respectively. The epoch threshold nₓ for introducing the pattern loss was set to 200, as detailed in Equation 39. Weighting coefficients for the loss function terms (L1, L2, and L3) were tuned to 1, 1e-4, and 0.1, respectively, to balance their influence on the total loss.

### 6.5.2. Synthesized Data

In this section, HarPE performance is studied in comparison to alternative methods, EMD, VMD, NMF, REPET and REPET-Extended, Spectral Masking, and DHF.

Dataset: the synthesized dataset presented in the DHF section of the present disclosure was utilized which contains five synthesized mixed biosensor signals generated under varying levels of interference. Each signal includes two or three sources, with some signals of interest contributing less than 10% of the total energy. The pulsation shapes are derived from the PhysioNet dataset [27], while the frequency and amplitude variations are manually crafted to include a wide range of interference scenarios.

PhysioNet dataset is citation [6]: Ary L Goldberger, Luis AN Amaral, Leon Glass, Jeffrey M Hausdorff, Plamen Ch Ivanov, Roger G Mark, Joseph E Mietus, George B Moody, Chung-Kang Peng, and HEugene Stanley. 2000. PhysioBank, PhysioToolkit, and PhysioNet: components of a new research resource for complex physiologic signals. circulation 101, 23 (2000), e215-e220.

Table 7 compares the studied signal separation methods, highlighting HarPE's significant improvements over alternative approaches, and reports Mean Square Error (MSE) and Signal to Distortion Ratio (SDR) metrics. To report the average, arithmetic mean is used in linear space for SDR, and geometric mean for MSE. On average, HarPE achieves 6.57 dB increase in SDR, and a 60% reduction in MSE compared to the best previous work. In scenarios where the target source energy is less than 10% of the mixed signal energy, specifically mixed signal 3 source 2; mixed signal 4 source 3; and mixed signal 5 source 3, HarPE outperforms all previous methods, showing a 21% MSE reduction and 1.58 dB SDR improvement compared to the best previous method.

Furthermore, HarPE significantly improves the stability of signal separation performance. To compare the SDR and MSE stability of estimated signals, we report Mean, Coefficient of Variation (CV), and Geometric Coefficient of Variation (GCV) as explained in Equations 43 through 44. In these equations, Z is a set of five separation results from applying the signal separation method to a target source five times.

$$CV ( Z ) = \frac{STD ( Z )}{Mean ( Z )} \qquad ( 43 )$$

$$GCV ( Z ) = \sqrt{{e}^{{STD ( ln ( Z ) )}^{2}} - 1} \qquad ( 44 )$$

Table 8 reports a notable reduction in SDR CV and MSE GCV when comparing HarPE with the state of the art, DHF method. HarPE improves the average SDR by 6.67 db, and average MSE error by 81%. While HarPE consistently improves the mean SDR and MSE results compared to DHF, it might show a wider performance variation in cases that the target signal becomes more difficult to separate due to long overlap or faint energy (e.g., mixed signal 3 source 2, and mixed signal 2 source 2).

### 6.5.3. Data Collected in Animal Experiments

Dataset: in-vivo data was utilized from eight Transabdominal Fetal Oximetry (TFO) sessions conducted on five pregnant ewes. The dataset includes mixed photoplethysmography (PPG) signals recorded at two 740 nm and 850 nm near infrared wavelengths, along with ground-truth blood gas oxygen saturation (SaO₂) values obtained from fetal blood draws at 3 to 5-minute intervals; which was described in the DHF section of this disclosure. To induce fetal hypoxia, the study uses an inflatable balloon catheter inserted in the ewe's aorta to control the blood flow to the uterus and fetus through which, fetal oxygen saturation level is regulated. PPG signals contain interferences from maternal heart and respiration pulsations, which must be isolated from fetal heart pulsations to estimate fetal oxygen saturation (SpO₂). Although the ground truth fetal signal is not accessible, we report improvements in the fetal SpO₂ estimation correlation with fetal SaO₂ readings after signal separation using HarPE compared to previous methods.

Fetal SpO₂ Estimation Method: the SpO₂ estimation technique was applied to the outputs of various signal separation methods. Initially, the pulsating and non-pulsating components of the separated fetal signal at both wavelengths are computed. Then the Ratio of Ratios (R) parameter is computed, as outlined in Equation 45. This ratio is used to estimate SpO₂, Y′Y′, through a parametric linear relationship with the inverse of SaO₂ labels, Y, as shown in Equation 46.

$$R = \frac{{( \frac{Pulsating}{Nonpulsating} Pulsating / Nonpulsating )}^{{λ}_{1}}}{{( \frac{Pulsating}{Nonpulsating} Pulsating / Nonpulsating )}^{{λ}_{2}}} \qquad ( 45 )$$

$${Y}^{′} = \frac{1}{Y + k} = {w}_{0} + {w}_{1} R , k = 1.885 {Y}^{′} = \frac{1}{Y + k} = {w}_{0} + {w}_{1} R , k = 1.885 \qquad ( 46 )$$

Then a detailed comparison was conducted of the correlation between the estimated SpO₂ values and the ground truth SaO₂ across individual experiment sessions.

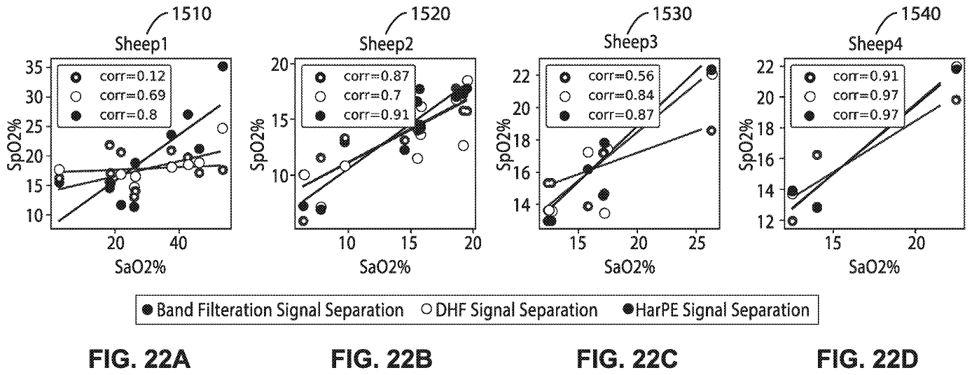

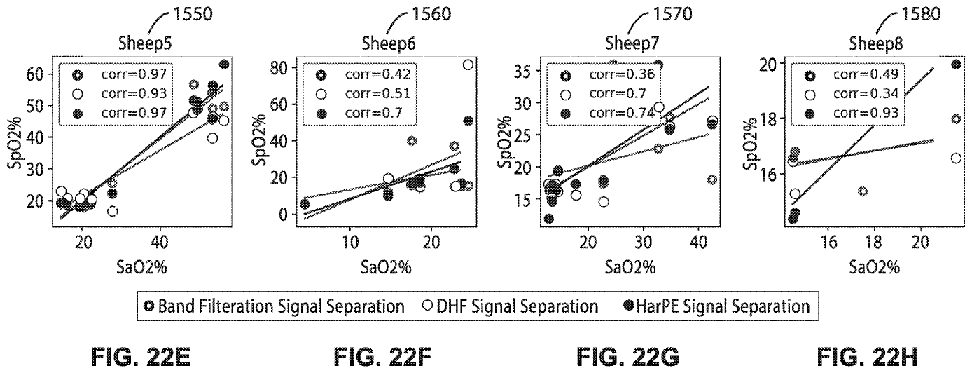

FIG. 22A through 22H illustrates a comparison of SpO₂ measurements for HarPE compared with DHF, and a Band Filtration method for signal sets Sheep1 through Sheep8 (1510, 1520, 1530, 1540, 1550, 1560, 1570, 1580). In these tests HarPE consistently outperformed the previous best methods of DHF and band filtration signal separation, by achieving closer approximations to the ideal correlation of one in every case. This demonstrates HarPE's superior accuracy in estimating fetal signal which improves the SaO₂ estimation accuracy under various experimental conditions. On average, HarPE improves the SpO₂-SaO₂ correlation by 55% and 40% when compared to band filtration and DHF technique respectively.

Moreover, HarPE enhances the overall stability of both the learning and signal separation processes, resulting in more consistent, high-quality signal separation performance compared to DHF. To demonstrate this with in-vivo data, the HarPE and DHF methods were run five times, each time with a different random initialization. Although the ground truth fetal signal was not directly available, the correlation between the ground truth SaO₂ and the estimated SpO₂ was evaluated after each run. The results, summarized in terms of mean and CV (refer to Equation 43) in Table 9, show that HarPE consistently improved the mean correlation. Additionally, the CV metric reflected a significant enhancement in most cases, indicating a more reliable and stable separation process. However, it is worth noting that the improvement was less pronounced in scenarios where the fundamental frequency measurements were noisy, leading to some deformation in the expected strictly periodic pattern. HarPE's overall performance marks a substantial advancement, delivering greater accuracy and stability in challenging conditions, with average improvement of 36% and 69% in mean correlation and CV, respectively.

### 6.6. Conclusion on HarPE

HarPE offers a significant advancement in signal separation for biosensors, effectively addressing the challenges of limited and noisy data with overlapping physiological signals. By integrating deep ensemble learning with a harmonic-aware deep prior model, HarPE enhances both the accuracy of signal isolation, and the learning stability.

### 7. General Scope of Embodiments

Embodiments of the technology of this disclosure may be described herein with reference to flowchart illustrations of methods and systems according to embodiments of the technology. Embodiments of the technology of this disclosure may also be described with reference to procedures, algorithms, steps, operations, formulae, or other computational depictions, which may be included within the flowchart illustrations or otherwise described herein. It will be appreciated that any of the foregoing may also be implemented as computer program instructions. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure(s) algorithm(s), step(s), operation(s), formula (e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, the terms controller, microcontroller, processor, microprocessor, hardware processor, computer processor, central processing unit (CPU), and computer are used synonymously to denote a device capable of executing the instructions and communicating with input/output interfaces and/or peripheral devices, and that the terms controller, microcontroller, processor, microprocessor, hardware processor, computer processor, CPU, and computer are intended to encompass single or multiple devices, single core and multicore devices, and variations thereof.

From the description herein, it will be appreciated that the present disclosure encompasses multiple implementations of the technology which include, but are not limited to, the following:

A method of separating non-stationary quasi-periodic signals from a mixture of signals, the method comprising: (a) performing, on a mixture of signals, multiple iterations of deep harmonic finesse (DHF) using a deep harmonic neural network in combination with a pattern alignment unit, wherein the neural network has a structure that embeds implicit harmonic priors within a time-frequency domain, wherein the pattern-alignment unit transforms the mixture of signals, and wherein a target non-stationary quasi-periodic signal is output from the neural network; (b) wherein each iteration of DHF receives, as data, an input signal comprising multi-source quasi-periodic signals, said multi-source quasi-periodic signals having a target signal received in an initial temporal space, said target signal having a target signal frequency, said multi-source quasi-periodic signals having a non-target signal frequency list; (c) wherein each iteration of DHF performs a pattern alignment operation based on the target signal frequency, wherein the pattern-alignment unit transforms the input signal into a new temporal space, wherein the target signal has a constant frequency; (d) wherein within each iteration of DHF, the pattern alignment operation is followed by a processing procedure for masking and in-painting a short-time Fourier transform (STFT) representation of the data, wherein a mask is used to conceal all significant spectral components of sources other than the target signal, and in-painting interpolates masked regions of the STFT representation of the data, wherein amplitude and phase are processed separately; and (e) wherein each iteration of DHF outputs a target signal to be subtracted from the input signal, and wherein a final DHF iteration outputs a target signal in which unwanted source signals have been removed.

A method of separating non-stationary quasi-periodic signals from a mixture of said signals, the method comprising: (a) performing multiple iterations of deep harmonic finesse (DHF); (b) wherein each iteration of DHF receives an input signal having multi-source quasi-periodic signals within which is a target signal as received in an initial temporal space, as well as a target frequency and a non-target frequency list, and outputs a target signal to be subtracted from the input signal; (c) wherein each iteration of DHF performs a pattern alignment operation based on the target signal frequency, in which pattern-alignment transforms the input signal into a new temporal space, in which the target signal has a constant frequency; (d) wherein within each iteration of DHF, pattern alignment is followed by a procedure for masking and in-painting a short-time Fourier transform (STFT) representation of data, wherein a mask is used to conceal all significant spectral components of sources other than the target signal, and in-painting interpolates the masked regions of STFT representation of data, handling amplitude and phase separately; and (e) utilizing prior knowledge of time-frequency patterns in the signals for in-painting masked spectrograms.

A method of separating non-stationary quasi-periodic signals from a mixture of signals, the method comprising: (a) performing multiple iterations of deep harmonic finesse (DHF) using a deep harmonic neural network coupled with a pattern alignment unit, in which the structure of the neural network embeds implicit harmonic priors within a time-frequency domain, while the pattern-alignment component transforms the mixture of signals, ensuring a strong alignment of a target non-stationary quasi-periodic signal with the neural network; (b) wherein each iteration of DHF receives an input signal, as data, having multi-source quasi-periodic signals having a target signal as received in an initial temporal space, and a target frequency and a non-target frequency list, and which outputs a target signal to be subtracted from the input signal; (c) wherein each iteration of DHF performs a pattern alignment operation based on the target signal frequency, wherein the pattern-alignment transforms the input signal into a new temporal space, wherein the target signal has a constant frequency; (d) wherein within each iteration of DHF, pattern alignment as performed in the pattern alignment unit is followed by a procedure for masking and in-painting a short-time Fourier transform (STFT) representation of data, wherein a mask is used to conceal all significant spectral components of sources other than the target signal, and in-painting interpolates the masked regions of the STFT representation of data, handling amplitude and phase separately; and (e) utilizing prior knowledge of time-frequency patterns in the multi-source quasi-periodic signals for in-painting masked spectrograms, wherein these signal transformations increase signal-to-distortion ratio of the target non-stationary quasi-periodic signal which is output from the deep harmonic neural network.

A method of separating non-stationary quasi-periodic signals from a mixture of signals, the method comprising: (a) performing multiple iterations of deep harmonic finesse (DHF) using a deep harmonic neural network coupled with a pattern alignment unit, in which the structure of the neural network embeds implicit harmonic priors within a time-frequency domain, while the pattern-alignment component transforms the mixture of signals, ensuring a strong alignment of a target non-stationary quasi-periodic signal with the neural network; (b) wherein each iteration of DHF receives an input signal, as data, having multi-source quasi-periodic signals having a target signal as received in an initial temporal space, and a target frequency, and a non-target frequency list and which outputs a target signal to be subtracted from the input signal; (c) wherein each iteration of DHF performs a pattern alignment operation based on the target signal frequency, wherein the pattern-alignment transforms the input signal into a new temporal space, wherein the target signal has a constant frequency; (d) wherein within each iteration of DHF, pattern alignment in the pattern alignment unit is followed by a procedure for masking and in-painting a short-time Fourier transform (STFT) representation of data, wherein a mask is used to conceal all significant spectral components of sources other than the target signal, and in-painting interpolates the masked regions of the STFT representation of data, handling amplitude and phase separately; (e) utilizing prior knowledge of time-frequency patterns in the signals for in-painting masked spectrograms; (f) wherein the pattern alignment component transforms the sensed signal, toward increasing alignment with the neural network; and (g) whereby the method utilizes prior knowledge of time-frequency patterns in the signals for masking and in-painting spectrograms increases signal-to-distortion ratio for non-stationary quasi-periodic signals.

An apparatus for separating non-stationary quasi-periodic signals, the apparatus comprising: (a) a processor configured to receive data corresponding to non-stationary quasi-periodic signals as an input; and (b) a non-transitory memory storing instructions executable by the processor as a deep harmonic neural network; (c) wherein said instructions, when executed by the processor, perform the steps comprising: (c) (i) separating non-stationary quasi-periodic signals in iterations of deep harmonic finesse (DHF) of the deep harmonic neural network coupled with a pattern alignment unit, in which the structure of the neural network embeds implicit harmonic priors within a time-frequency domain, while the pattern-alignment component transforms the mixture of signals, ensuring a strong alignment of a target non-stationary quasi-periodic signal with the neural network; (c) (ii) wherein each iteration of DHF receives an input signal, as data, having multi-source quasi-periodic signals having a target signal as received in an initial temporal space, and a target frequency, and a non-target frequency list and which outputs a target signal to be subtracted from the input signal; (c) (iii) wherein each iteration of DHF performs a pattern alignment operation based on the target signal frequency, wherein the pattern-alignment transforms the input signal into a new temporal space, and wherein the target signal has a constant frequency; (c)(iv) wherein within each iteration of DHF, pattern alignment, in the pattern alignment unit is followed by a deep prior learning procedure for masking and in-painting a short-time Fourier transform (STFT) representation of data, wherein a mask is used to conceal all significant spectral components of sources other than the target signal, and in-painting interpolates the masked regions of the STFT representation of data, handling amplitude and phase separately; and (c)(v) utilizing prior knowledge of time-frequency patterns in the signals for in-painting masked spectrograms, wherein these signal transformations increase signal-to-distortion ratio of the target non-stationary quasi-periodic signal which is output from the deep harmonic neural network.

An apparatus for separating non-stationary quasi-periodic signals, the apparatus comprising: (a) a processor configured to receive data corresponding to non-stationary quasi-periodic signals as an input; and (b) a non-transitory memory storing instructions executable by the processor; (c) wherein said instructions, when executed by the processor, perform the method of any preceding or following implementation.

A method of separating non-stationary quasi-periodic signals, the method comprising: performing multiple rounds of deep harmonic finesse (DHF), for separation of non-stationary quasi-periodic signals in iterations; wherein each round of DHF receives an input signal, a target frequency, and non-target frequency list and outputs a target signal to be subtracted from the input signal; wherein each round of DHF performs a pattern alignment operation based on the target signal frequency, wherein pattern-alignment resamples and transforms the input signal into a new space, providing the network with an input in which the target signal has a constant frequency; wherein within each round of DHF, pattern alignment is followed by a deep prior learning procedure for masking and in-painting a short-time Fourier transform (STFT) representation of data, wherein a mask is used to conceal all significant spectral components of sources other than the selected one, and in-painting interpolates the masked regions of the selected signal, targeting amplitude and phase separately, to recover values during spectral overlaps and crossover; and whereby the method utilizing prior knowledge of time-frequency patterns in the signals for masking and in-painting spectrograms increased signal-to-distortion ratio for non-stationary quasi-periodic signals.

The method or apparatus of any preceding or following implementation, wherein utilizing prior knowledge of time-frequency patterns in the signals to in-paint spectrograms is performed through a deep harmonic neural network coupled with an integrated pattern alignment component.

The method or apparatus of any preceding or following implementation, wherein the pattern alignment component transforms the sensed signal, toward increasing alignment with the neural network.

The method or apparatus of any preceding or following implementation, wherein said method is utilized for collecting physiological data subject to being impacted by quasi-periodic non-stationary artifacts.

The method or apparatus of any preceding or following implementation, wherein the input signals are from single sensor measurements, and signal fundamental frequencies are known, but not their amplitudes, either through auxiliary sensing modalities or preliminary analysis of the mixed signal.

The method or apparatus of any preceding or following implementation, wherein said method overcomes potential overlap of signal frequencies which limit the performance of conventional frequency-based filtering techniques, and the limitations of having only a single sample, which precludes the use of traditional machine learning methods that require a training dataset.

The method or apparatus of any preceding or following implementation, wherein said utilizing prior knowledge of time-frequency patterns in the signals to in-paint spectrograms is performed through a deep harmonic neural network coupled with an integrated pattern alignment component.

The method or apparatus of any preceding or following implementation, wherein the pattern alignment component transforms the sensed signal, ensuring a strong alignment with the neural network.

The method or apparatus of any preceding or following implementation, wherein said method is utilized for collecting physiological data which is otherwise impacted by quasi-periodic non-stationary artifacts.

The method or apparatus of any preceding or following implementation, wherein the input signals are from single sensor measurements, and signal fundamental frequencies, but not their amplitudes, are known either through auxiliary sensing modalities or preliminary analysis of the mixed signal.

The method or apparatus of any preceding or following implementation, wherein said method overcomes potential overlap of signal frequencies which renders classic frequency-based filtering techniques ineffective, and the limitations of having only a single sample, which precludes the use of traditional machine learning methods that require a training dataset.

The method or apparatus of any preceding or following implementation, wherein the non-stationary quasi-periodic signals are obtained from a single sensor source.

A method of separating non-stationary quasi-periodic signals from a mixture of signals, the method comprising: (a) performing multiple iterations of deep harmonic finesse (DHF) on a neural network receiving a mixture of signals having multiple signal sources; (b) wherein said DHF comprises a pattern alignment operation, a masking operation, a short-time Fourier transform (STFT) interpolation operation, and a pattern restoration operation; (c) wherein each iteration of DHF receives, as data, an input signal comprising multi-source quasi-periodic signals containing multiple unwanted source signals which are targeted for removal at unwanted target frequencies; (d) wherein each DHF iteration receives a non-target signal frequency list; (e) wherein each DHF iteration outputs an unwanted source signal as its target signal for the iteration; (f) wherein said unwanted source signals are removed within each iteration of DHF by performing a pattern alignment operation based on the target signal frequency for the unwanted source signal, and wherein the pattern alignment operation transforms the input signal into a new temporal space; (g) wherein the pattern alignment operation is followed by a masking operation in which non-target frequencies of a spectral representation of the mixture of signals are masked out leaving only a target of the unwanted source signal; (h) wherein the masking operation is based on the target signal frequency and non-target signal frequency list to conceal all signal sources except the unwanted source signal for the iteration; (i) wherein the masking operation is followed by an STFT interpolation operation which performs deep spectral in-painting and cyclic phase interpolation; (j) wherein said in-painting uses spectrally accurate harmonic convolution to in-paint the masked non-target signals and recover the target signal of the unwanted source signal for the iteration; (k) wherein the in-painting is followed by cyclic phase interpolation in which phase information is extracted for each DHF iteration from unwarped STFT complex values, both real and imaginary and using a previously generated mask, the phase is estimated and interpolated over time, and the results of the cyclic phase interpolation are integrated to create an unwarped target signal, upon which a pattern restoration operation is performed; and (l) wherein for each DHF iteration, specific targets for unwanted source signals are subtracted from the input signal received by the iteration and passed to a subsequent DHF iteration, and wherein a final DHF iteration outputs a target signal in which said unwanted source signals have been removed.

A method for signal separation in multi-source quasi-periodic signals, the method comprising: (a) selecting a mixed signal for separation, said mixed signal having an original mixed signal space; (b) performing a short-time Fourier transform (STFT) on the mixed signal; (c) masking and in-painting the STFT; (d) wherein said masking conceals spectral components of signal sources other than the selected signal; (e) wherein said in-painting interpolates masked regions of the selected signal by targeting amplitude and phase separately to recover values during spectral overlaps and crossover; (f) wherein said in-painting employs a signal pattern aligner and a convolutional neural network; (g) wherein the pattern aligner resamples and unwarps the mixed signal, and transforms the mixed signal into a space where the selected mixed signal is strictly periodic; (h) wherein the convolutional neural network defines a neighborhood in time and frequency by dilation and accessing integral multiples of a target bin, respectively; (i) performing phase interpolation by separately interpolating real and imaginary parts of each frequency bin's phase over time and recalculating the interpolated phase; and (j) performing an inverse short time Fourier transform (ISTFT) on results of the in-painting and interpolated phase information to transform the mixed signal to the original mixed signal space and providing a signal source separated from the mixed signal.

The method or apparatus of any preceding implementation, further comprising subtracting the separated signal source from the mixed signal to produce a residual for use as a signal source for further signal separation.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these groups of elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system, or method.

As used herein, the term “set” refers to a collection of one or more objects. Thus, for example, a set of objects can include a single object or multiple objects.

Relational terms such as first and second, top and bottom, upper and lower, left and right, and the like, may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, apparatus, or system, that comprises, has, includes, or contains a list of elements does not include only those elements but may include other elements not expressly listed or inherent to such process, method, article, apparatus, or system. An element proceeded by “comprises . . . a”, “has a”, “includes . . . a”, “contains . . . a” does not, without more constraints, preclude the existence of additional identical elements in the process, method, article, apparatus, or system, that comprises, has, includes, contains the element.

As used herein, the terms “approximately”, “approximate”, “substantially”, “substantial”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential feature or element of the technology described herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after the application is filed. Accordingly, the reader should consult the application as filed for the original content of the disclosure. Any deletion of content of the disclosure should not be construed as a disclaimer, forfeiture, or dedication to the public of any subject matter of the application as originally filed.

All text in a drawing figure is hereby incorporated into the disclosure and is to be treated as part of the written description of the drawing figure.

The following claims are hereby incorporated into the disclosure, with each claim standing on its own as a separately claimed subject matter.

Although the description herein contains many details, these should not be construed as limiting the scope of the disclosure, but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

The following is a list of publications referenced in the description above using numbers inside square brackets (e.g., [1]) and which are used for comparisons in the tables below. Each publication is incorporated herein by reference in its entirety.

### Empirical Mode Decomposition (EMD):

[1] Norden E Huang, Zheng Shen, Steven R Long, Manli C Wu, Hsing H Shih, Quanan Zheng, Nai-Chyuan Yen, Chi Chao Tung, and Henry H Liu. 1998. The empirical mode decomposition and the Hilbert spectrum for nonlinear and non-stationary time series analysis. Proceedings of the Royal Society of London. Series A: mathematical, physical and engineering sciences 454, 1971 (1998), 903-995.

### Variational Mode Decomposition (VMD):

[2] Konstantin Dragomiretskiy and Dominique Zosso. 2013. Variational mode decomposition. IEEE transactions on signal processing 62, 3 (2013), 531-544.

### Non-Negative Matrix Factorization (NMF):

[3] Daniel D Lee and H Sebastian Seung. 1999. Learning the parts of objects by non-negative matrix factorization. Nature 401, 6755 (1999), 788-791.

### REpeating Pattern Extraction Technique (REPET) & REPET-Extended:

[4] Zafar Rafiiand Bryan Pardo.2012. Repeating pattern extraction technique (REPET): A simple method for music/voice separation. IEEE transactions on audio, speech, and language processing 21, 1 (2012), 73-84.

### Spectral Masking:

[5] Timo Gerkmann and Emmanuel Vincent. 2018. Spectral masking and filtering. Audio source separation and speech enhancement (2018), 65-85.

### PhysioNet Dataset:

[6] Ary L Goldberger, Luis AN Amaral, Leon Glass, Jeffrey M Hausdorff, Plamen Ch Ivanov, Roger G Mark, Joseph E Mietus, George B Moody, Chung-Kang Peng, and HEugene Stanley. 2000. PhysioBank, PhysioToolkit, and PhysioNet: components of a new research resource for complex physiologic signals. circulation 101, 23 (2000), e215-e220.

TABLE 1
Overview of the Synthesized Mixed Signals

| | | Synthesized Mixed Signals | | | | |
|---|---|---|---|---|---|---|
| | | MSig1 | MSig2 | MSig3 | MSig4 | MSig5 |
| source1 | mean(A) | 0.08 | 0.08 | 0.4 | 0.74 | 0.6 |
| | std(A) | 0.02 | 0.01 | 0.1 | 0.1 | 0.2 |
| | f_min | 0.9 | 0.8 | 1.4 | 0.5 | 0.5 |
| | f_max | 1.7 | 1.2 | 2.3 | 0.9 | 0.9 |
| source2 | mean(A) | 0.03 | 0.06 | 0.03 | 0.08 | 0.07 |
| | std(A) | 0.01 | 0.02 | 0.01 | 0.01 | 0.01 |
| | f_min | 1.8 | 1.0 | 1.6 | 1.1 | 1.0 |
| | f_max | 3.0 | 2.1 | 3.0 | 1.8 | 2.0 |
| source3 | mean(A) | - | - | - | 0.06 | 0.04 |
| | std(A) | - | - | - | 0.01 | 0.01 |
| | f_min | - | - | - | 1.8 | 2.1 |
| | f_max | - | - | - | 2.9 | 3.5 |
| noise | mean | 0.0 | 0.0 | 0.0 | 0.0 | 0.0 |
| | std | 0.003 | 0.01 | 0.04 | 0.01 | 0.001 |

TABLE 2
Performance Comparison of Signal Separation Methods
with Best Performance per Source Separation is Bolded

| | | | | | | | | | | REPET- | | Spect. | | | |
|---|---|---|---|---|---|---|---|---|---|---|---|---|---|---|---|
| | | EMD [1] | | VMD [2] | | NMF [3] | | REPET [4] | | Ext. [4] | | Masking [5] | | DHF | |
| | | SDR | | SDR | | SDR | | SDR | | SDR | | SDR | | SDR | |
| | | (db) | MSE | (db) | MSE | (db) | MSE | (db) | MSE | (db) | MSE | (db) | MSE | (db) | MSE |
| MSig | s1 | −1.38 | 7.4e−4 | 7.32 | 1.5e−4 | −9.03 | 8.9e−4 | 4.68 | 2.0e−4 | 9.91 | 1.0e−4 | 12.31 | 6.4e−5 | 21.63 | 7.4e−6 |
| 1 | s2 | −6.17 | 1.3e−4 | 3.17 | 1.1e−4 | −7.53 | 1.3e−4 | -0.77 | 6.4e−05 | -10.82 | 1.1e−4 | 6.44 | 3.3e−5 | 15.51 | 4.1e−6 |
| MSig | s1 | −6.36 | 9.1e−4 | 3.14 | 7.1e−4 | -4.58 | 7.8e−4 | 0.09 | 4.8e−4 | 4.82 | 3.4e−4 | 4.51 | 3.5e−4 | 9.29 | 1.1e−4 |
| 2 | s2 | −21.75 | 7.2e−4 | -21.06 | 7.0e−4 | -4.98 | 6.4e−4 | -1.25 | 4.5e−4 | -6.2 | 4.4e−4 | 1.16 | 5.6e−4 | 9.02 | 9.2e−5 |
| MSig | s1 | 5.65 | 5.3e−3 | 7.24 | 3.9e−3 | -8.79 | 2.2e−2 | 6.59 | 3.3e−3 | 14.36 | 8.1e−4 | 26.95 | 5.7e−5 | 21.18 | 2.1e−4 |
| 3 | s2 | 0.07 | 2.6e−4 | -0.15 | 1.8e−4 | -0.18 | 8.3e−4 | -0.04 | 2.7e−4 | -1.63 | 2.1e−4 | -17.3 | 9.9e−3 | 6.96 | 4.0e−5 |
| MSig | s1 | 5.2 | 1.1e−2 | 15.16 | 1.5e−3 | -4.95 | 3.6e−2 | 3.83 | 9.9e−3 | 18.19 | 7.8e−4 | 23.81 | 2.2e−4 | 28.86 | 6.9e−5 |
| 4 | s2 | 0.36 | 9.5e−4 | 0.76 | 8.7e−4 | -2.63 | 1.0e−3 | -0.11 | 9.3e−4 | -4.29 | 6.0e−4 | 4.03 | 3.8e−4 | 14.25 | 3.7e−5 |
| | s3 | −13.79 | 4.0e−4 | -19.95 | 4.0e−4 | -5.59 | 4.6e−4 | -15.76 | 3.9e−4 | -7.26 | 3.2e−4 | 8.9 | 5.3e−5 | 14.7 | 3.3e−5 |
| MSig | s1 | 2.11 | 1.6e−2 | 15.53 | 1.1e−3 | -4.31 | 2.6e−2 | 1.26 | 1.1e−2 | 18.81 | 5.2e−4 | 19.26 | 4.2e−4 | 23.97 | 1.4e−4 |
| 5 | s2 | −5.27 | 7.4e−4 | 1.02 | 7.0e−4 | -5.64 | 7.2e−4 | -0.05 | 7.3e−4 | -4.42 | 4.3e−4 | 1.27 | 5.5e−4 | 14.48 | 2.6e−5 |
| | s3 | −18.59 | 1.2e−4 | 3.01 | 1.1e−4 | -10.47 | 1.2e−4 | -11.59 | 1.2e−4 | -7.82 | 1.0e−4 | 6.82 | 2.7e−5 | 15.06 | 5.1e−6 |

| | | | | | | | | | | | | | | |
|---|---|---|---|---|---|---|---|---|---|---|---|---|---|---|
| Average | 0.10 | 9.5e−4 | 8.69 | -4.84 | -4.84 | 1.4e−3 | 1.49 | 6.7e−4 | 11.86 | 3.2e−4 | 18.56 | 2.1e−5 | 20.88 | 3.6e−5 |

TABLE 3
Performance Comparison of Signal Separation Methods on source-specific filtered
synthesized mixed signals, with Best Performance per Source Separation is Bolded.

| | | EMD | | VMD | | NMF | | REPET | | REPET- | | Spect. | | | |
|---|---|---|---|---|---|---|---|---|---|---|---|---|---|---|---|
| | | [1] | | [2] | | [3] | | [4] | | Ext. [4] | | Masking [5] | | DHF | |
| | | SDR | | SDR | | SDR | | SDR | | SDR | | SDR | | SDR | |
| | | (db) | MSE | (db) | MSE | (db) | MSE | (db) | MSE | (db) | MSE | (db) | MSE | (db) | MSE |
| MSig1 | s1 | 7.45 | 1.8e−4 | 0.85 | 1.0e−3 | -8.98 | 8.9e−4 | 5.03 | 1.6e−4 | 11.01 | 6.8e−0.5 | 12.31 | 6.4e−5 | 21.92 | 7.0e−6 |
| | s2 | 2.21 | 7.1e−0.5 | 1.49 | 1.4e−4 | -8.24 | 1.2e−4 | 3.37 | 3.2e−0.5 | 4.83 | 3.1e−0.5 | 6.44 | 3.3e−5 | 18.30 | 2.6e−6 |
| MSig2 | s1 | 2.6 | 4.5e−4 | 1.83 | 1.0e−3 | -5.3 | 7.7e−4 | 1.74 | 3.1e−4 | 4.92 | 2.7e−4 | 4.51 | 3.5e−4 | 12.37 | 5.7e−5 |
| | s2 | 1.17 | 4.9e−4 | 1.1 | 7.5e−4 | -5.49 | 5.7e−4 | 0.81 | 3.5e−4 | 2.58 | 4.2e−4 | 1.16 | 5.6e−4 | 11.30 | 5.3e−5 |
| MSig3 | s1 | 1.26 | 9.9e−3 | 13.7 | 1.0e−3 | -8.73 | 2.2e−2 | 6.62 | 2.9e−3 | 16.08 | 5.3e−4 | 26.95 | 5.7e−5 | 15.81 | 7.2e−4 |
| | s2 | 0.04 | 1.8e−4 | 0.04 | 3.1e−4 | -0.25 | 6.9e−4 | 0.02 | 2.0e−4 | 0.06 | 1.8e−4 | -17.3 | 9.9e−3 | 7.79 | 3.0e−5 |
| MSig4 | s1 | 4.07 | 1.4e−2 | 9.71 | 6.1e−3 | -4.96 | 3.6e−2 | 9.54 | 3.6e−3 | 18.29 | 6.7e−4 | 23.81 | 2.2e−4 | 30.23 | 5.0e−5 |
| | s2 | 2.74 | 4.5e−4 | 0.27 | 1.4e−3 | -7.06 | 7.6e−4 | 2.16 | 2.8e−4 | 4.95 | 2.6e−4 | 4.03 | 3.8e−4 | 13.12 | 4.7e−5 |
| | s3 | 7.19 | 7.3-0.5 | -5.3 | 2.7e−4 | -9.11 | 3.5e−4 | 4.49 | 6.5e−0.5 | 6.43 | 5.5e−0.5 | 8.9 | 5.3e−5 | 14.37 | 1.5e−5 |
| MSig5 | s1 | 0.7 | 1.9e−2 | 14.43 | 1.4e−3 | -4.32 | 2.6e−2 | 6.26 | 4.8e−3 | 18.5 | 5.0e−4 | 19.26 | 4.2e−4 | 24.69 | 1.3e−4 |
| | s2 | 0.83 | 4.9e−4 | 3.53 | 4.5e−4 | -5.2 | 6.2e−4 | 1.07 | 2.9e−4 | 3.42 | 3.0e−4 | 1.27 | 5.5e−4 | 18.35 | 1.1e−5 |
| | s3 | 5.64 | 3.7e−0.5 | 0.43 | 1.2e−4 | -8.73 | 1.1e−4 | 2.82 | 2.7e−0.5 | 5.06 | 2.3e−0.5 | 6.82 | 2.7e−5 | 11.51 | 8.7e−6 |

| | | | | | | | | | | | | | | |
|---|---|---|---|---|---|---|---|---|---|---|---|---|---|---|
| Average | 3.74 | 5.5e−4 | 7.76 | 6.5e−4 | -5.51 | 1.2e−3 | 4.59 | 3.1e−4 | 12.50 | 1.7e−4 | 18.56 | 2.1e−4 | 21.71 | 3.0e−5 |

TABLE 4
Overview of Synthesized Mixed Signals

| | | MSig1 | MSig2 | MSig3 | MSig4 | MSig5 |
|---|---|---|---|---|---|---|
| source1 | mean(A) | 0.08 | 0.08 | 0.4 | 0.74 | 0.6 |
| | std(A) | 0.02 | 0.01 | 0.1 | 0.1 | 0.2 |
| | fmin | 0.9 | 0.8 | 1.4 | 0.5 | 0.5 |
| | fmax | 1.7 | 1.2 | 2.3 | 0.9 | 0.9 |
| source2 | mean(A) | 0.03 | 0.06 | 0.03 | 0.08 | 0.07 |
| | std(A) | 0.01 | 0.02 | 0.01 | 0.01 | 0.01 |
| | fmin | 1.8 | 1.0 | 1.6 | 1.1 | 1.0 |
| | fmax | 3.0 | 2.1 | 1.8 | 1.8 | 2.0 |
| source3 | mean(A) | -- | -- | -- | 0.06 | 0.04 |
| | std(A) | -- | -- | -- | 0.01 | 0.01 |
| | fmin | -- | -- | -- | 1.8 | 2.1 |
| | fmax | -- | -- | -- | 2.9 | 3.5 |
| noise | mean | 0.0 | 0.0 | 0.0 | 0.0 | 0.0 |
| | std | 0.003 | 0.01 | 0.04 | 0.01 | 0.001 |

TABLE 5
Performance Comparison of Signal Separation Methods on source-specific filtered
synthesized mixed signals, with Best Performance per Source Separation is Bolded.

| | | | | | | |
|---|---|---|---|---|---|---|
| | | | | REPET- | Spect. | |
| EMD [1] | VMD [2] | NMF [3] | REPET [4] | Ext. [4] | Masking [5] | DHF |

| | | SDR | | SDR | | SDR | | SDR | | SDR | | SDR | | SDR | |
|---|---|---|---|---|---|---|---|---|---|---|---|---|---|---|---|
| | | (db) | MSE | (db) | MSE | (db) | MSE | (db) | MSE | (db) | MSE | (db) | MSE | (db) | MSE |
| MSig1 | s1 | -1.38 | 7.4e−4 | 7.32 | 1.5e−4 | -9.03 | 8.9e−4 | 4.68 | 2.0e−4 | 9.91 | 1.0e−4 | 12.31 | 6.4e−5 | 21.92 | 7.0e−6 |
| | s2 | -6.17 | 1.3e−4 | 3.17 | 1.1e−4 | -7.53 | 1.3e−4 | -0.77 | 6.4e−0.5 | -10.82 | 1.1e−4 | 6.44 | 3.3e−5 | 18.30 | 2.6e−6 |
| MSig2 | s1 | -6.36 | 9.1e−4 | 3.14 | 7.1e−4 | -4.58 | 7.8e−4 | 0.09 | 4.8e−4 | 4.82 | 3.4e−4 | 4.51 | 3.5e−4 | 12.37 | 5.7e−5 |
| | s2 | -21.75 | 7.2e−4 | -21.06 | 7.0e−4 | -4.98 | 6.4e−4 | -1.25 | 4.5e−4 | -6.2 | 4.4e−4 | 1.16 | 5.6e−4 | 11.30 | 5.3e−5 |
| MSig3 | s1 | 5.65 | 5.3e−3 | 7.24 | 3.9e−3 | -8.79 | 2.2e−2 | 6.59 | 3.3e−3 | 14.36 | 8.1e−4 | 26.95 | 5.7e−5 | 15.81 | 7.2e−4 |
| | s2 | 0.07 | 2.6e−4 | -0.15 | 1.8e−4 | -0.18 | 8.3e−4 | -0.04 | 2.7e−4 | -1.63 | 2.1e−4 | -17.3 | 9.9e−3 | 7.79 | 3.0e−5 |
| MSig4 | s1 | 5.2 | 1.1e−2 | 15.16 | 1.5e−3 | -4.95 | 3.6e−2 | 3.83 | 9.9e−3 | 18.19 | 7.8e−4 | 23.81 | 2.2e−4 | 30.23 | 5.0e−5 |
| | s2 | 0.36 | 9.5e−4 | 0.76 | 8.7e−4 | -2.63 | 1.0e−3 | -0.11 | 9.3e−4 | -4.29 | 6.0e−4 | 4.03 | 3.8e−4 | 13.12 | 4.7e−5 |
| | s3 | -13.79 | 4.0e−4 | -19.95 | 4.0e−4 | -5.59 | 4.6e−4 | -15.64 | 3.9e−4 | -7.26 | 3.2e−4 | 8.9 | 5.3e−5 | 14.37 | 1.5e−5 |
| MSig5 | s1 | 2.11 | -1.6e−2 | 15.53 | 1.1e−3 | -4.31 | 2.6e−2 | 1.26 | 1.1e−2 | 18.81 | 5.2e−4 | 19.26 | 4.2e−4 | 24.69 | 1.3e−4 |
| | s2 | -5.27 | 7.4e−4 | 1.02 | 7.0e−4 | -5.64 | 7.2e−4 | -0.05 | 7.3e−4 | -4.42 | 4.3e−4 | 1.27 | 5.5e−4 | 18.53 | 1.1e−5 |
| | s3 | -18.59 | 1.2e−4 | 3.01 | 1.1e−4 | -10.47 | 1.2e−4 | -11.59 | 1.2e−4 | -7.82 | 1.0e−4 | 6.82 | 2.7e−5 | 11.51 | 8.7e−6 |

| | | | | | | | | | | | | | | |
|---|---|---|---|---|---|---|---|---|---|---|---|---|---|---|
| Average | 0.10 | 9.5e−4 | 8.69 | 5.0e−4 | -4.84 | 1.4e−3 | 1.49 | 6.7e−4 | 11.86 | 3.2e−4 | 18.56 | 2.1e−4 | 21.71 | 3.0e−5 |

TABLE 6
Performance Comparison of Signal Separation Methods on source-specific filtered
synthesized mixed signals, with Best Performance per Source Separation is Bolded.

| | | | | | | | | | | REPET- | | Spect. | | | |
|---|---|---|---|---|---|---|---|---|---|---|---|---|---|---|---|
| | | EMD [1] | | VMD [2] | | NMF [3] | | REPET [4] | | Ext. [4] | | Masking [5] | | DHF | |
| | | SDR | | SDR | | SDR | | SDR | | SDR | | SDR | | SDR | |
| | | (db) | MSE | (db) | MSE | (db) | MSE | (db) | MSE | (db) | MSE | (db) | MSE | (db) | MSE |
| MSig | s1 | 7.45 | 1.8e−4 | 0.85 | 1.0e−3 | -8.98 | 8.9e−4 | 5.03 | 1.6e−4 | 11.01 | 6.8e−0.5 | 12.31 | 6.4e−5 | 21.19 | 7.0e−6 |
| 1 | s2 | 2.21 | 7.1e−0.5 | 1.49 | 1.4e−4 | -8.24 | 1.2e−4 | 3.37 | 3.2e−0.5 | 4.83 | 3.1e−0.5 | 6.44 | 3.3e−5 | 18.30 | 2.6e−6 |
| MSig | s1 | 2.6 | 4.5e−4 | 1.83 | 1.0e−3 | -5.3 | 7.7e−4 | 1.74 | 3.1e−4 | 4.92 | 2.7e−4 | 4.51 | 3.5e−4 | 12.37 | 5.7e−5 |
| 2 | s2 | 1.17 | 4.9e−4 | 1.1 | 7.5e−4 | -5.49 | 5.7e−4 | 0.81 | 3.5e−4 | 2.58 | 4.2e−4 | 1.16 | 5.6e−4 | 11.30 | 5.3e−5 |
| MSig | s1 | 1.26 | 9.9e−3 | 13.7 | 1.0e−3 | -8.73 | 2.2e−2 | 6.62 | 2.9e−3 | 16.08 | 5.3e−4 | 26.95 | 5.7e−5 | 15.81 | 7.2e−4 |
| 3 | s2 | 0.04 | 1.8e−4 | 0.04 | 3.1e−4 | -0.25 | 6.9e−4 | 0.02 | 2.0e−4 | 0.06 | 1.8e−4 | -17.3 | 9.9e−3 | 7.79 | 3.0e−5 |
| MSig | s1 | 4.07 | 1.4e−2 | 9.71 | 6.1e−3 | -4.96 | 3.6e−2 | 9.54 | 3.6e−3 | 18.29 | 6.7e−4 | 23.81 | 2.2e−4 | 30.23 | 5.0e−5 |
| 4 | s2 | 2.74 | 4.5e−4 | 0.27 | 1.4e−3 | -7.06 | 7.6e−4 | 2.16 | 2.8e−4 | 4.95 | 2.6e−4 | 4.03 | 3.8e−4 | 13.12 | 4.7e−5 |
| | s3 | 7.19 | 7.3e−0.5 | -5.3 | 2.7e−4 | -9.11 | 3.5e−4 | 4.49 | 6.5e−0.5 | 6.43 | 5.5e−0.5 | 8.9 | 5.3e−5 | 14.37 | 1.5e−5 |
| MSig | s1 | 0.7 | 1.9e−2 | 14.43 | 1.4e−3 | -4.32 | 2.6e−2 | 6.26 | 4.8e−3 | 18.5 | 5.0e−4 | 19.26 | 4.2e−4 | 24.69 | 1.3e−4 |
| 5 | s2 | 0.83 | 4.9e−4 | 3.53 | 4.5e−4 | -5.2 | 6.2e−4 | 1.07 | 2.9e−4 | 3.42 | 3.0e−4 | 1.27 | 5.5e−4 | 18.35 | 1.1e−5 |
| | s3 | 5.64 | 3.7e−0.5 | 0.43 | 1.2e−4 | -8.73 | 1.1e−4 | 2.82 | 2.7e−0.5 | 5.06 | 2.3e−0.5 | 6.82 | 2.7e−5 | 11.51 | 8.7e−6 |

| | | | | | | | | | | | | | | |
|---|---|---|---|---|---|---|---|---|---|---|---|---|---|---|
| Average | 3.74 | 5.5e−4 | 7.76 | 6.5e−4 | -5.51 | 1.2e−3 | 4.59 | 3.1e−4 | 12.50 | 1.7e−4 | 18.56 | 2.1e−4 | 21.71 | 3.0e−5 |

TABLE 7
Performance Comparison with HarPE
(Best Performance per Source Separation Bolded)

| | | | | | | | | | | REPET- | | Spect. | | | | | |
|---|---|---|---|---|---|---|---|---|---|---|---|---|---|---|---|---|---|
| | | EMD [1] | | VMD [2] | | NMF [3] | | REPET [4] | | Ext. [4] | | Masking[5] | | DHF | | HarPE | |
| | | SDR | | SDR | | SDR | | SDR | | SDR | | SDR | | SDR | | SDR | |
| | | (db) | MSE | (db) | MSE | (db) | MSE | (db) | MSE | (db) | MSE | (db) | MSE | (db) | MSE | (db) | MSE |
| MSig1 | s1 | -1.38 | 7.4e−4 | 7.32 | 1.5e−4 | -9.03 | 8.9e−4 | 4.68 | 2.0e−4 | 9.91 | 1.0e−4 | 12.31 | 6.4e−5 | 21.92 | 7.0e−6 | 25.81 | 2.9e−6 |
| | s2 | -6.17 | 1.3e−4 | 3.17 | 1.1e−4 | -7.53 | 1.3e−4 | -0.77 | 6.4e−05 | -10.82 | 1.1e−4 | 6.44 | 3.3e−5 | 18.30 | 2.6e−6 | 21.49 | 1.3e−6 |
| MSig2 | s1 | -6.36 | 9.1e−4 | 3.14 | 7.1e−4 | -4.58 | 7.8e−4 | 0.09 | 4.8e−4 | 4.82 | 3.4e−4 | 4.51 | 3.5e−4 | 12.37 | 5.7e−5 | 13.19 | 4.7e−5 |
| | s2 | -21.75 | 7.2e−4 | -21.06 | 7.0e−4 | -4.98 | 6.4e−4 | -1.25 | 4.5e−4 | -6.2 | 4.4e−4 | 1.16 | 5.6e−4 | 11.30 | 5.3e−5 | 14.41 | 2.6e−5 |
| MSig3 | s1 | 5.65 | 5.3e−3 | 7.24 | 3.9e−3 | -8.79 | 2.2e−2 | 6.59 | 3.3e−3 | 14.36 | 8.1e−4 | 26.95 | 5.7e−5 | 15.81 | 7.2e−4 | 26.43 | 6.2e−5 |
| | s2 | 0.07 | 2.6e−4 | -0.15 | 1.8e−4 | -0.18 | 8.3e−4 | -0.04 | 2.7e−4 | -1.63 | 2.1e−4 | -17.3 | 9.9e−3 | 7.79 | 3.0e−5 | 8.98 | 2.3e−5 |
| MSig4 | s1 | 5.2 | 1.1e−2 | 15.16 | 1.5e−3 | -4.95 | 3.6e−2 | 3.83 | 9.9e−3 | 18.19 | 7.8e−4 | 23.81 | 2.2e−4 | 30.23 | 5.0e−5 | 34.36 | 1.9e−5 |
| | s2 | 0.36 | 9.5e−4 | 0.76 | 8.7e−4 | -2.63 | 1.0e−3 | -0.11 | 9.3e−4 | -4.29 | 6.0e−4 | 4.03 | 3.8e−4 | 13.12 | 4.7e−5 | 14.07 | 3.7e−5 |
| | s3 | -13.79 | 4.0e−4 | -19.95 | 4.0e−4 | -5.59 | 4.6e−4 | -15.76 | 3.9e−4 | -7.26 | 3.2e−4 | 8.9 | 5.3e−5 | 14.37 | 1.5e−5 | 15.11 | 1.2e−5 |
| MSig5 | s1 | 2.11 | 1.6e−2 | 15.53 | 1.1e−3 | -4.31 | 2.6e−2 | 1.26 | 1.1e−2 | 18.81 | 5.2e−4 | 19.26 | 4.2e−4 | 24.69 | 1.3e−4 | 35.96 | 1.0e−5 |
| | s2 | -5.27 | 7.4e−4 | 1.02 | 7.0e−4 | -5.64 | 7.2e−4 | -0.05 | 7.3e−4 | -4.42 | 4.3e−4 | 1.27 | 5.5e−4 | 18.53 | 1.1e−5 | 24.69 | 2.5e−5 |
| | s3 | -18.59 | 1.2e−4 | 3.01 | 1.1e−4 | -10.47 | 1.2e−4 | -11.59 | 1.2e−4 | -7.82 | 1.0e−4 | 6.82 | 2.7e−5 | 11.51 | 8.7e−6 | 14.09 | 4.8e−6 |

| | | | | | | | | | | | | | | | | |
|---|---|---|---|---|---|---|---|---|---|---|---|---|---|---|---|---|
| Average | 0.10 | 9.5e−4 | 8.69 | -4.84 | -4.84 | 1.4e−3 | 1.49 | 6.7e−4 | 11.86 | 3.2e−4 | 18.56 | 2.1e−5 | 21.71 | 3.0e−5 | 28.28 | 1.2e−5 |

TABLE
Stability of HarPE vs. DHF observed in five optimization repetition

| | | DHF | | HarPE | | DHF | | HarPE | |
|---|---|---|---|---|---|---|---|---|---|
| | | Mean | CV | Mean | CV | GMean | GCV | GMean | GCV |
| MSig1 | s1 | 18.81 | 0.99 | 25.80 | 0.19 | 2.1e−5 | 1.31 | 2.9e−6 | 0.19 |
| | s2 | 16.46 | 1.16 | 21.96 | 0.08 | 6.7e−6 | 1.55 | 1.1e−6 | 0.08 |
| MSig2 | s1 | 10.74 | 0.48 | 14.05 | 0.39 | 9.3e−5 | 0.63 | 4.1e−5 | 0.38 |
| | s2 | 6.47 | 0.22 | 12.62 | 0.42 | 1.7e−4 | 0.23 | 4.3e−5 | 0.51 |
| MSig3 | s1 | 9.32 | 1.01 | 26.52 | 0.69 | 4.6e−3 | 1.15 | 7.1e−5 | 0.61 |
| | s2 | 5.86 | 0.15 | 6.92 | 0.37 | 4.7e−5 | 0.16 | 3.8e−5 | 0.36 |
| MSig4 | s1 | 19.02 | 0.52 | 34.30 | 0.10 | 7.4e−4 | 0.59 | 2.0e−5 | 0.10 |
| | s2 | 13.36 | 0.14 | 13.86 | 0.12 | 4.5e−5 | 0.15 | 4.0e−5 | 0.13 |
| | s3 | 11.57 | 0.43 | 14.28 | 0.31 | 3.0e−5 | 0.37 | 1.6e−5 | 0.35 |
| MSig5 | s1 | 21.77 | 1.03 | 35.01 | 0.24 | 4.2e−4 | 1.56 | 1.3e−5 | 0.25 |
| | s2 | 16.74 | 1.05 | 19.32 | 0.09 | 2.5e−5 | 1.48 | 1.5e−5 | 0.23 |
| | s3 | 7.71 | 0.51 | 13.20 | 0.33 | 2.3e−5 | 0.56 | 6.2e−6 | 0.40 |

| | | | | | | | | |
|---|---|---|---|---|---|---|---|---|
| Average | 13.15 | 0.64 | 19.82 | 0.28 | 8.4e−5 | 0.60 | 1.6e−5 | 0.25 |

(Note - Mean and GMean in dB. * GMean: * CV and *GCV defined in the Equations)

TABLE 9
Correlation Stability of fetal SpO₂ vs. fetal SaO₂

| | |
|---|---|
| DHF | HarPE |

| | Mean | CV | Mean | CV |
|---|---|---|---|---|
| Sheep1 | 0.37 | 0.62 | 0.71 | 0.03 |
| Sheep2 | 0.56 | 0.31 | 0.71 | 0.03 |
| Sheep3 | 0.46 | 0.41 | 0.64 | 0.36 |
| Sheep4 | 0.82 | 0.32 | 0.86 | 0.04 |
| Sheep5 | 0.74 | 0.34 | 0.94 | 0.04 |
| Sheep6 | 0.33 | 0.55 | 046 | 0.26 |
| Sheep7 | 0.59 | 0.23 | 0.62 | 0.12 |
| Sheep8 | 0.20 | 0.54 | 0.56 | 0.15 |
| Average | 0.51 | 0.42 | 0.69 | 0.13 |

(Note - Mean and GMean in dB. *GMean: *CV and *GCV defined in the Equations

1. A method of separating non-stationary quasi-periodic signals from a mixture of signals, the method comprising:
(a) performing, on a mixture of signals, multiple iterations of deep harmonic finesse (DHF) using a deep harmonic neural network in combination with a pattern alignment unit, wherein the neural network has a structure that embeds implicit harmonic priors within a time-frequency domain, wherein the pattern-alignment unit transforms the mixture of signals, and wherein a target non-stationary quasi-periodic signal is output from the neural network;
(b) wherein each iteration of DHF receives, as data, an input signal comprising multi-source quasi-periodic signals, the multi-source quasi-periodic signals having a target signal received in an initial temporal space, the target signal having a target signal frequency, the multi-source quasi-periodic signals having a non-target signal frequency list;
(c) wherein each iteration of DHF performs a pattern alignment operation based on the target signal frequency, wherein the pattern-alignment unit transforms the input signal into a new temporal space, wherein the target signal has a constant frequency;
(d) wherein within each iteration of DHF, the pattern alignment operation is followed by a processing procedure for masking and in-painting a short-time Fourier transform (STFT) representation of the data, wherein a mask is used to conceal all significant spectral components of sources other than the target signal, and in-painting interpolates masked regions of the STFT representation of the data, wherein amplitude and phase are processed separately; and
(e) wherein each iteration of DHF outputs a target signal to be subtracted from the input signal, and wherein a final DHF iteration outputs a target signal in which unwanted source signals have been removed.

2. The method of claim 1, wherein utilizing prior knowledge of time-frequency patterns in the signals to in-paint spectrograms is performed through a deep harmonic neural network coupled with an integrated pattern alignment component.

3. The method of claim 2, wherein the pattern alignment component transforms the input signal, toward increasing alignment with the neural network.

4. The method of claim 1, wherein the method is utilized for collecting data subject to being impacted by quasi-periodic non-stationary artifacts.

5. The method of claim 1, wherein the input signal is from single sensor measurements, and signal fundamental frequencies are known, but not their amplitudes, either through auxiliary sensing modalities or preliminary analysis of the mixed signal.

6. The method of claim 1, wherein the method overcomes potential overlap of signal frequencies which limit performance of conventional frequency-based filtering techniques, and limitations of having only a single sample, which preclude use of traditional machine learning methods that require a training dataset.

7. A method of separating non-stationary quasi-periodic signals from a mixture of signals, the method comprising:
(a) performing multiple iterations of deep harmonic finesse (DHF) using a deep harmonic neural network coupled with a pattern alignment unit, in which the structure of the neural network embeds implicit harmonic priors within a time-frequency domain, while the pattern-alignment component transforms the mixture of signals, ensuring a strong alignment of a target non-stationary quasi-periodic signal with the neural network;
(b) wherein each iteration of DHF receives an input signal, as data, having multi-source quasi-periodic signals having a target signal as received in an initial temporal space, and a target frequency, and a non-target frequency list and which outputs a target signal to be subtracted from the input signal;
(c) wherein each iteration of DHF performs a pattern alignment operation based on the target signal frequency, wherein the pattern-alignment transforms the input signal into a new temporal space, wherein the target signal has a constant frequency;
(d) wherein within each iteration of DHF, pattern alignment in the pattern alignment unit is followed by a procedure for masking and in-painting a short-time Fourier transform (STFT) representation of data, wherein a mask is used to conceal all significant spectral components of sources other than the target signal, and in-painting interpolates the masked regions of the STFT representation of data, handling amplitude and phase separately;
(e) utilizing prior knowledge of time-frequency patterns in the signals for in-painting masked spectrograms;
(f) wherein the pattern alignment component transforms the sensed signal, toward increasing alignment with the neural network; and
(g) whereby the method utilizes prior knowledge of time-frequency patterns in the signals for masking and in-painting spectrograms increases signal-to-distortion ratio for non-stationary quasi-periodic signals.

8. The method of claim 7, wherein the method is utilized for collecting data subject to being impacted by quasi-periodic non-stationary artifacts.

9. The method of claim 7, wherein the input signal is from single sensor measurements, and signal fundamental frequencies are known, but not their amplitudes, either through auxiliary sensing modalities or preliminary analysis of the mixed signal.

10. The method of claim 7, wherein the method overcomes potential overlap of signal frequencies which limit performance of conventional frequency-based filtering techniques, and limitations of having only a single sample, which preclude use of traditional machine learning methods that require a training dataset.

11. An apparatus for separating non-stationary quasi-periodic signals, the apparatus comprising:
(a) a processor configured to receive data corresponding to non-stationary quasi-periodic signals as an input; and
(b) a non-transitory memory storing instructions executable by the processor as a deep harmonic neural network;
(c) wherein the instructions, when executed by the processor, perform the steps comprising:
(i) separating non-stationary quasi-periodic signals in iterations of deep harmonic finesse (DHF) of the deep harmonic neural network coupled with a pattern alignment unit, in which the structure of the neural network embeds implicit harmonic priors within a time-frequency domain, while the pattern-alignment component transforms the mixture of signals, ensuring a strong alignment of a target non-stationary quasi-periodic signal with the neural network;
(ii) wherein each iteration of DHF receives an input signal, as data, having multi-source quasi-periodic signals having a target signal as received in an initial temporal space, and a target frequency, and a non-target frequency list and which outputs a target signal to be subtracted from the input signal;
(iii) wherein each iteration of DHF performs a pattern alignment operation based on the target signal frequency, wherein the pattern-alignment transforms the input signal into a new temporal space, and wherein the target signal has a constant frequency;
(iv) wherein within each iteration of DHF, pattern alignment, in the pattern alignment unit is followed by a deep prior learning procedure for masking and in-painting a short-time Fourier transform (STFT) representation of data, wherein a mask is used to conceal all significant spectral components of sources other than the target signal, and in-painting interpolates the masked regions of the STFT representation of data, handling amplitude and phase separately; and
(v) utilizing prior knowledge of time-frequency patterns in the signals for in-painting masked spectrograms, wherein these signal transformations increase signal-to-distortion ratio of the target non-stationary quasi-periodic signal which is output from the deep harmonic neural network.

12. The apparatus of claim 11, wherein the utilizing prior knowledge of time-frequency patterns for in-painting masked spectrograms is performed through the deep harmonic neural network coupled with the pattern alignment component.

13. The apparatus of claim 11, wherein the pattern alignment component transforms the input signal, toward increasing alignment with the neural network.

14. The apparatus of claim 11, wherein the apparatus is utilized for collecting data subject to being impacted by quasi-periodic non-stationary artifacts.

15. The apparatus of claim 14, wherein the data comprises physiological data.

16. The apparatus of claim 11, wherein the input signals are from single sensor measurements, and signal fundamental frequencies are known, but not their amplitudes, either through auxiliary sensing modalities or preliminary analysis of the mixed signals.

17. The apparatus of claim 11, wherein the apparatus overcomes potential overlap of signal frequencies which limit performance of conventional frequency-based filtering techniques, and limitations of having only a single sample, which precludes use of traditional machine learning methods that require a training dataset.

18-20. (canceled)
